(19) United States
(12) Patent Application Publication
(10) Pub. No.: US 2003/0109476 A1
(43) Pub. Date: Jun. 12, 2003

(54) Compositions and methods for the prevention and treatment of Huntington's disease

(76) Inventors: Eric B. Kmiec, Landenberg, PA (US); Hetal Parekh-Olmedo, Mantua, NJ (US)

Correspondence Address: FISH & NEAVE, 1251 AVENUE OF THE AMERICAS, 50TH FLOOR, NEW YORK, NY, 10020-1105, US

(21) Appl. No.: 10/215,432

(22) Filed: Aug. 7, 2002

Related U.S. Application Data
(60) Provisional application No. 60/310,757, filed on Aug. 7, 2001.
(60) Provisional application No. 60/310,889, filed on Aug. 8, 2001.
(60) Provisional application No. 60/310,770, filed on Aug. 8, 2001.
(60) Provisional application No. 60/337,219, filed on Dec. 4, 2001.

(51) Int. Cl. A61K 48/00; A61K 38/16; C07H 21/04; A61K 31/675; C07K 14/00

(52) U.S. Cl. 514/44; 514/81; 514/12; 530/350; 536/23.2

(57) ABSTRACT

The present invention provides compositions and methods for the prevention and treatment of a neurodegenerative disease, specifically Huntington's disease. In particular, the invention provides single-stranded, modified oligonucleotides for the targeted alteration of the genetic sequence of the Huntington's disease gene, and mehods of treating or preventing Huntington's disease using the same.

### CROSS REFERENCE TO RELATED APPLICATIONS

This application claims the benefit of U.S. provisional application serial No. 60/310,757, filed Aug. 7, 2001; No. 60/310,889, filed Aug. 8, 2001; No. 60/310,770, filed Aug. 8, 2001; and No. 60/337,219, filed Dec. 4, 2001, the disclosures of which are incorporated herein by reference in their entireties.

### TECHNICAL FIELD OF THE INVENTION

The present invention provides methods and compositions for the prevention and treatment of a neurodegenerative disease, specifically Huntington's disease.

### BACKGROUND

The concept of using synthetic oligonucleotides to alter DNA sequence directly within cells has matured throughout the last decade, with a variety of approaches having been explored with various degrees of success.

In one approach, triplex-forming oligonucleotides have been used. These oligonucleotides form a third strand within the double helix to direct nucleotide exchange in episomes and chromosomes. See, e.g., Chan et al., J. Biol Chem 274:11541-48 (1999), and references therein. But triplex-forming oligonucleotides have significant sequence constraints: target sequences must be polypurine rich to enable stable triple helix formation.

Given the target sequence requirements of the triplex-forming oligonucleotides, synthetic oligonucleotides that have more sequence versatility have been developed.

Chimeric RNA/DNA oligonucleotides have been shown to have more liberal target sequence requirements than triplexing oligonucleotides. The correcting oligonucleotide is a linear RNA/DNA chimera, structured into a double-stranded, double-hairpin configuration. See, e.g., Kmiec E B, Gene Therapy 6:1-3 (1999). See also U.S. Pat. No. 5,505,350 (the disclosure of which is hereby incorporated by reference in its entirety). Early experiments with such oligonucleotides demonstrated gene repair through nucleotide exchange in episomal (Yoon et al., Proc. Natl. Acad. Sci. USA,93:2071-2076 (1996)) and chromosomal systems (Cole-Strauss et al., Science 273:1386-1389 (1996)).

Although less constrained by target sequence than triplexing oligonucleotides, the chimeric oligonucleotides are difficult to synthesize and may exhibit only moderate gene correcting activity.

Recently, Kmiec and colleagues identified a simpler, single-stranded, oligonucleotide molecular structure whose activity in nucleotide exchange rivals and even surpasses that of chimeric RNA/DNA oligonucleotides (see WO 01/73002, the disclosure of which is hereby incorporated by reference). This molecular structure is a modified single stranded oligonucleotide.

Although triplexing, chimeric, and modified single-stranded oligonucleotides have been shown to be effective in mediating targeted gene alteration, it has not previously been known whether even the more liberal target sequence requirements of chimeric double-hairpin and modified single stranded oligonucleotides would permit targeting and alteration of highly repetitive sequences, such as the expanded triplet repeats characteristic of Huntington's disease.

Huntington's disease (“HD”) is a neurodegenerative disease characterized by abnormal protein aggregation.

Huntington's disease is a devastating autosomal dominant, fully penetrant, neurodegenerative disease resulting from a single mutation in the gene. The HD gene has been isolated (the human HD gene is on chromosome 4P16.3) and the mutation has been found. The mutation is an expansion of a trinucleotide repeat (CAG) in exon 1 of the HD gene, resulting in a polyglutamine (poly-Q) expansion in the protein (called Huntingtin). The resulting “gain of function” is the basis for the pathological, clinical and cellular sequelae of Huntington's Disease.

Neuropathologically, the most striking changes occur in the caudate nucleus and putamen, where the medium spiny neurons are particularly vulnerable.

Clinically, Huntington's disease is characterized by an involuntary choreiform movement disorder, psychiatric and behavioral changes and dementia. The age of onset is usually between the thirties and fifties, although juvenile and late onset cases of HD occur.

At the cellular level, Huntington's disease is characterized by protein aggregation in the cytoplasm and nucleus of neurons. Further examination of the protein aggregates revealed that the aggregates comprise ubiquitinated terminal fragments of Huntingtin. In human cells, ubiquitinated proteins or protein fragments are degraded by the proteasome system. There is accumulating evidence that the proteasome degradation system does not properly clear protein aggregates in diseases such as Huntington's Disease. Furthermore, the protein aggregates may themselves cause the proteasome to malfunction. See, e.g., Bence et al., Science 292: pp. 1552-1555 (2001). See also Waelter et al., Molecular Biology of the Cell 12: pp. 1393-1407 (2001).

For Huntington's diseases, genetic tests now permit the identification of individuals destined to develop HD from an at-risk population, making possible early intervention, even prior to the onset of neuronal degeneration or clinical symptoms.

Although several molecular approaches for gene therapy of HD have been investigated at the DNA, RNA and protein levels (reviewed in Constantini et al., Gene Therapy 7:93-109 (2000)), there is currently no effective treatment or preventive measure for HD: no therapeutic agent exists for HD and no means of prevention exists. Anti-sense strategies have not been shown to be effective therapy.

There thus exists a need for approaches that will delay, prevent, and/or treat the signs and/or symptoms of Huntington's disease.

### SUMMARY OF THE INVENTION

This invention solves these and other needs in the art by providing compositions and methods for treating Huntington's disease.

This invention provides an oligonucleotide for the targeted alteration of the genetic sequence of the Huntington's disease gene, said oligonucleotide comprising a single-stranded oligonucleotide having a DNA domain having at least one mismatch with respect to the genetic sequence of the Huntington's disease gene to be altered; and said oligonucleotide further comprising chemical modifications of the oligonucleotide, said chemical modifications being selected from the group consisting of an o-methyl modification, a “locked nucleic acid” (“LNA”) modification including LNA derivatives and analogs, two or more phosphorothioate linkages on a terminus, and a combination of any two or more of these modifications.

This invention also provides an oligonucleotide for targeted alteration of the genetic sequence of the Huntington's disease gene, comprising a chimeric RNA/DNA oligonucleotide, said oligonucleotide having at least one mismatch with respect to the genetic sequence of the Huntington's disease gene to be altered.

This invention further provides methods of using the above-described oligonucleotides for the targeted alteration of the genetic material of the Huntington's disease gene. This invention also provides methods of using the above-identified oligonucleotides to prevent or treat Huntington's disease, as well as methods of using the above-described oligonucleotides to cause disaggregation or to inhibit the formation of Huntingtin comprising protein aggregates, which are characteristic of Huntington's disease.

This invention also provides methods of treating or preventing Huntington's disease, as well as methods of causing disaggregation of or inhibiting the formation of Huntingtin comprising protein aggregates, which are characteristics of Huntington's disease, comprising administering to a subject an effective amount of an oligonucleotide that does or does not hybridize to the HD gene.

The foregoing and other objects, features and advantages of the present invention, as well as the invention itself, will be more fully understood from the following description of preferred embodiments.

### BRIEF DESCRIPTION OF THE DRAWINGS

FIG. 1 is a portion of the genomic sequence of a wild type allele of the human HD gene. Only the DNA sequence of the HD gene exon 1 and DNA sequences immediately upstream and immediately downstream of HD gene exon 1 are shown. The amino acid sequence of the human HD gene exon 1 is also shown. The DNA encoding HD gene exon 1 is clearly marked. This DNA is a wild type allele of the human HD gene. Allelic variations have been shown to exist in the population. * denotes where HD gene exon 1 starts and ** denotes where HD gene exon 1 ends. This allele is obtained from the human genome project sequencing effort and is deposited under accession number NT_{—}006081.

FIG. 2(a) is the amino acid sequence of HD gene exon 1, as derived from FIG. 1, and the DNA encoding that sequence, also as derived from FIG. 1. The amino acid sequence shows that the poly Q stretch can be a number n, wherein n is any number equal to or greater than 1. When n is 20, the Huntingtin exon 1 is wild type. The DNA sequence shows that the poly Q is due to an expansion of the codon that specifies glutamine. That codon can be either CAG or CAA.

* Any of the CAG can also be CAA.

# The wild type has approximately 20 CAG/CAA.

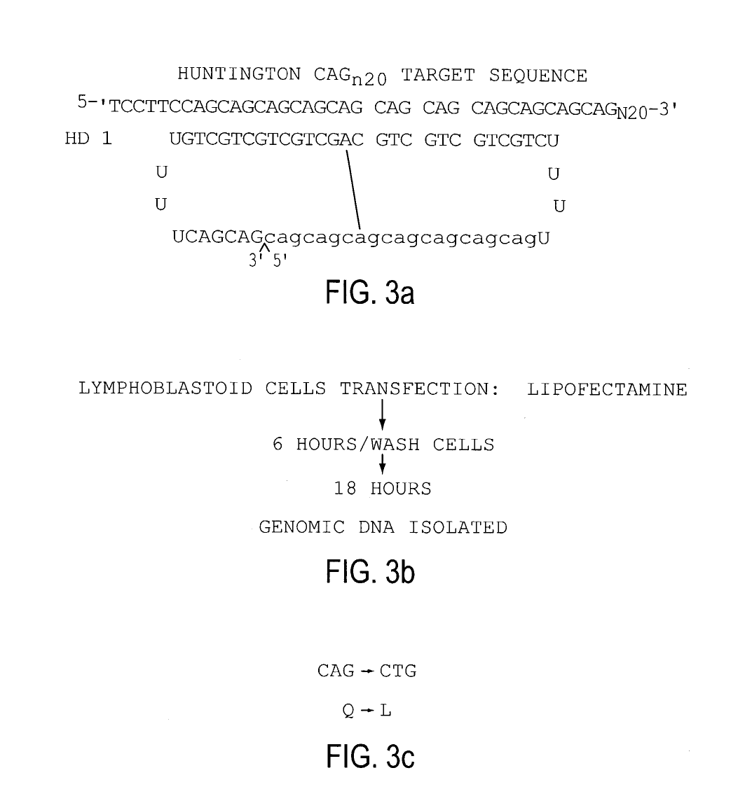

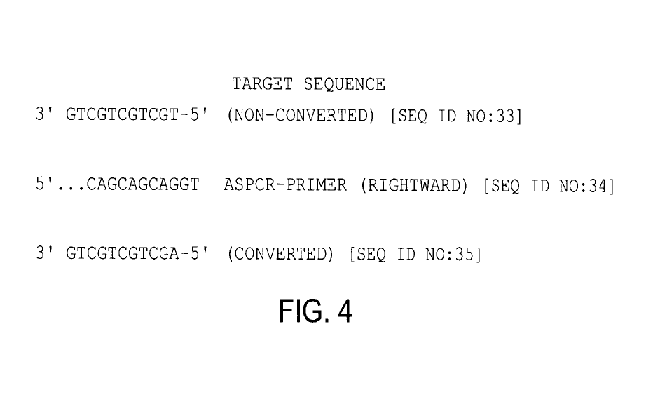

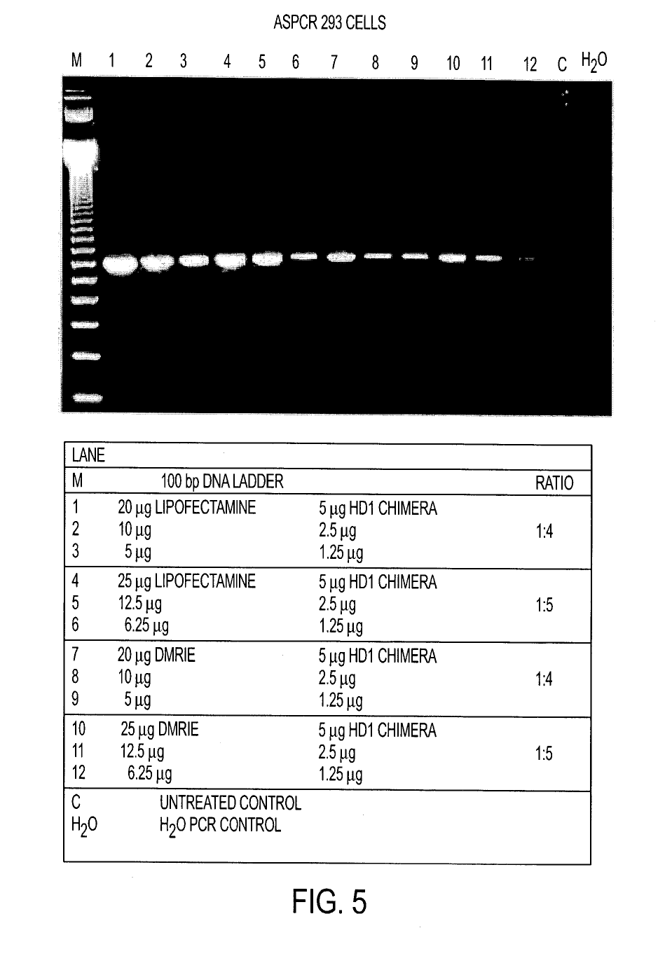

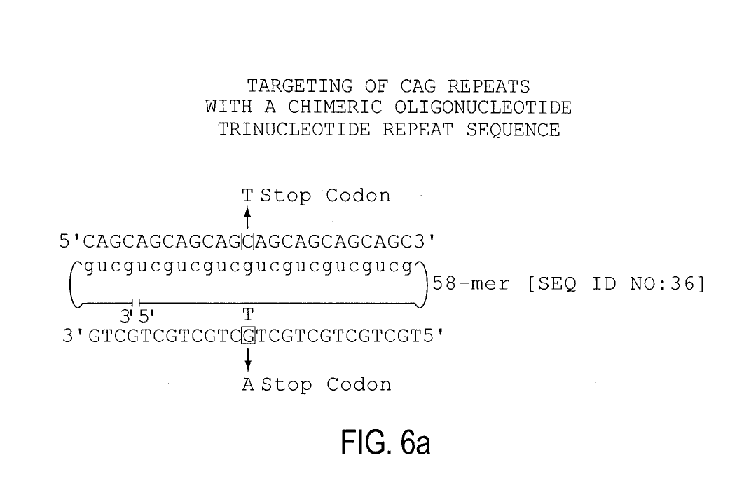

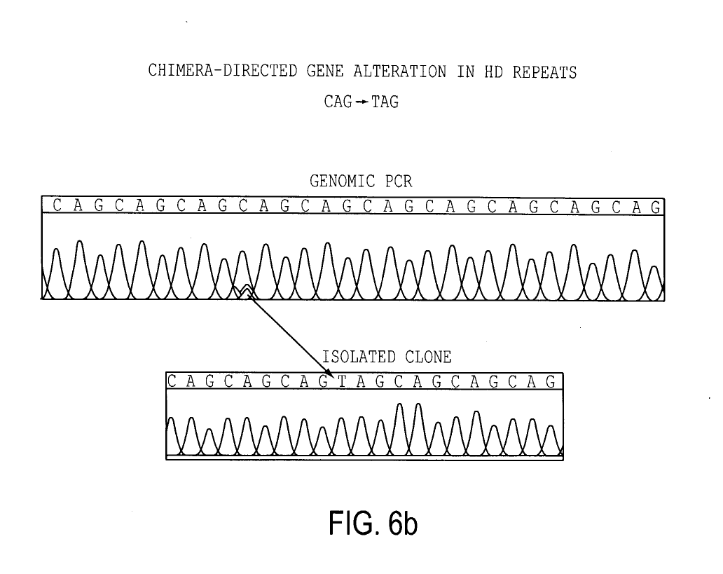

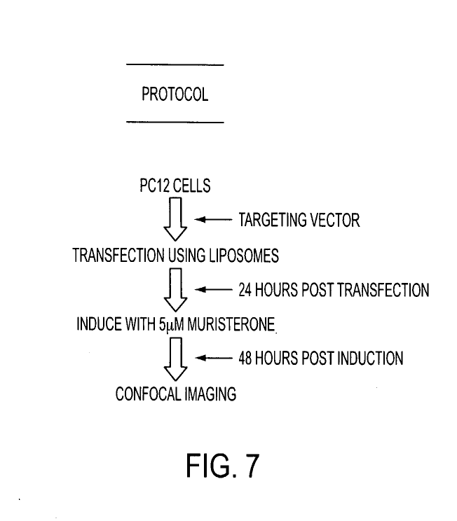

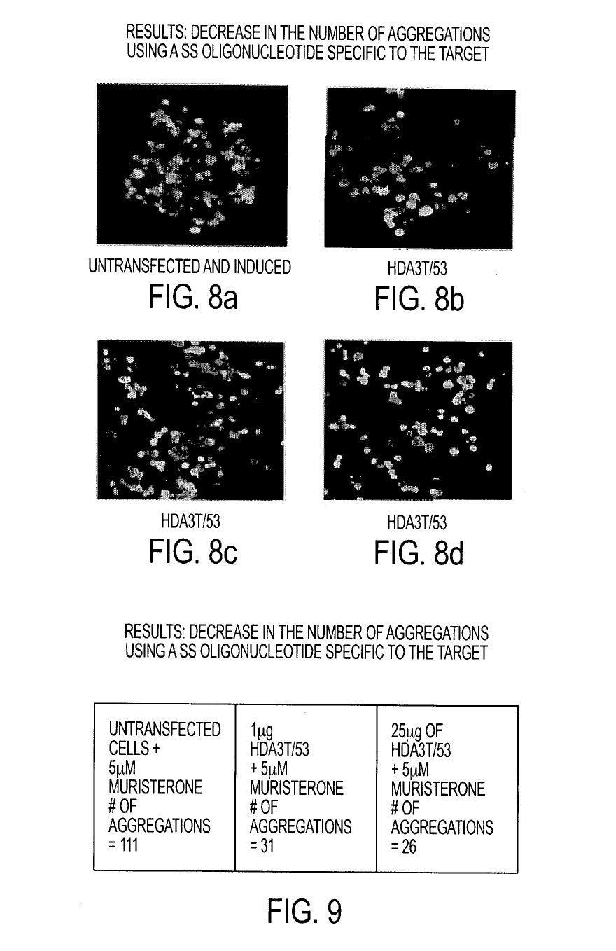

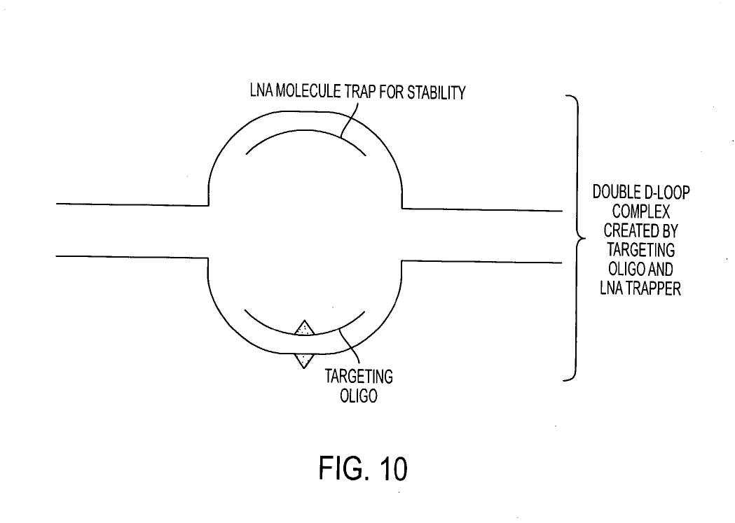

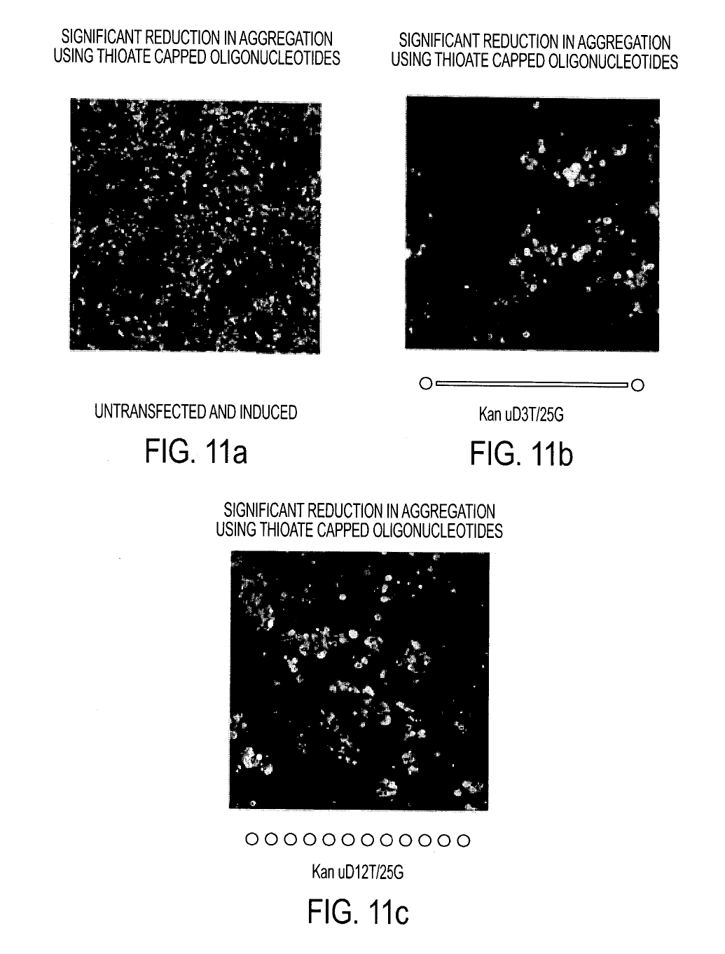

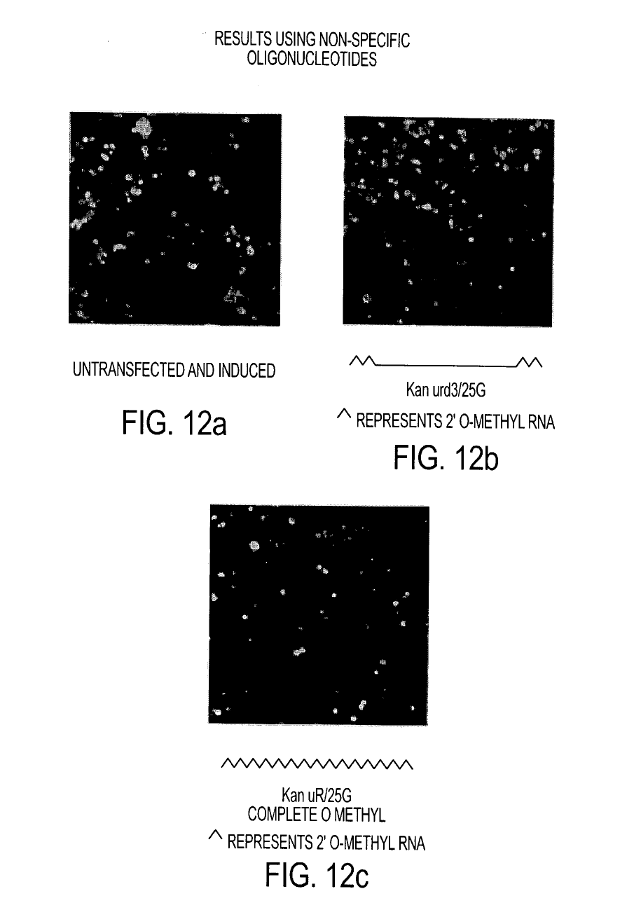

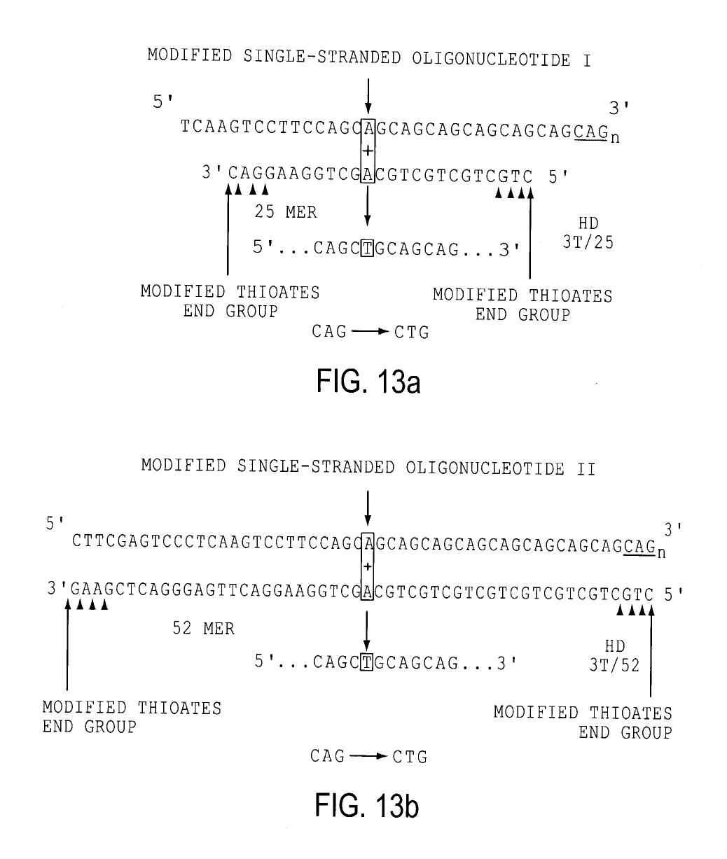

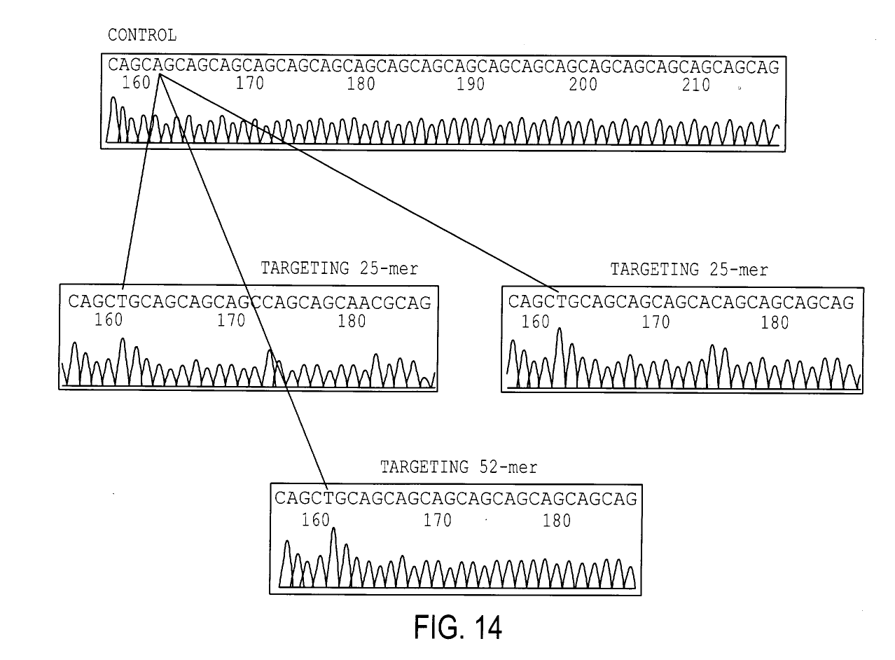

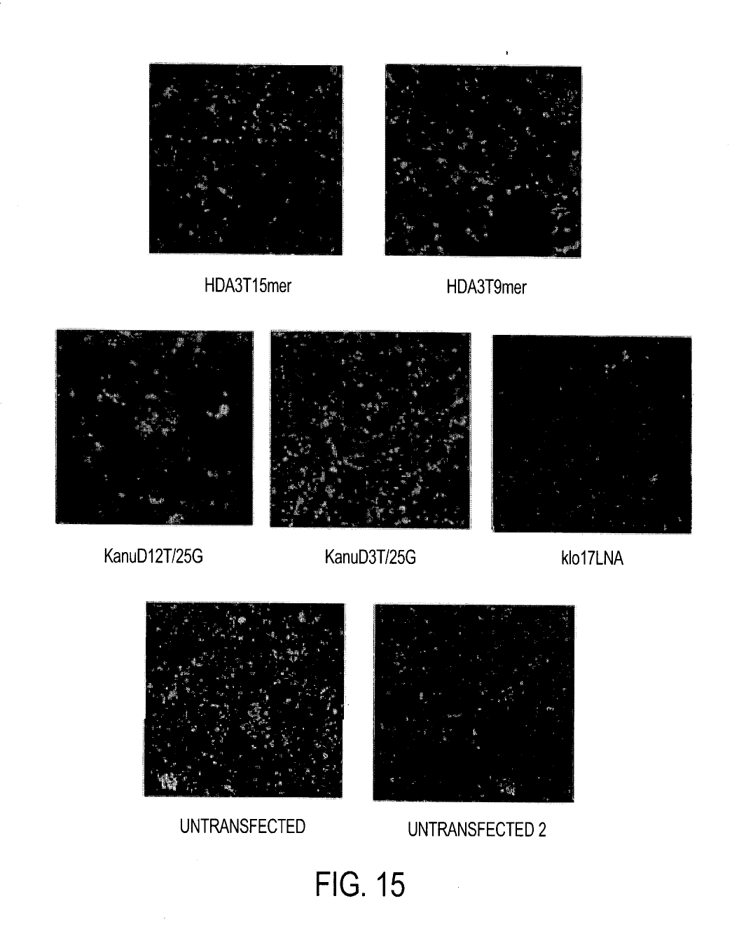

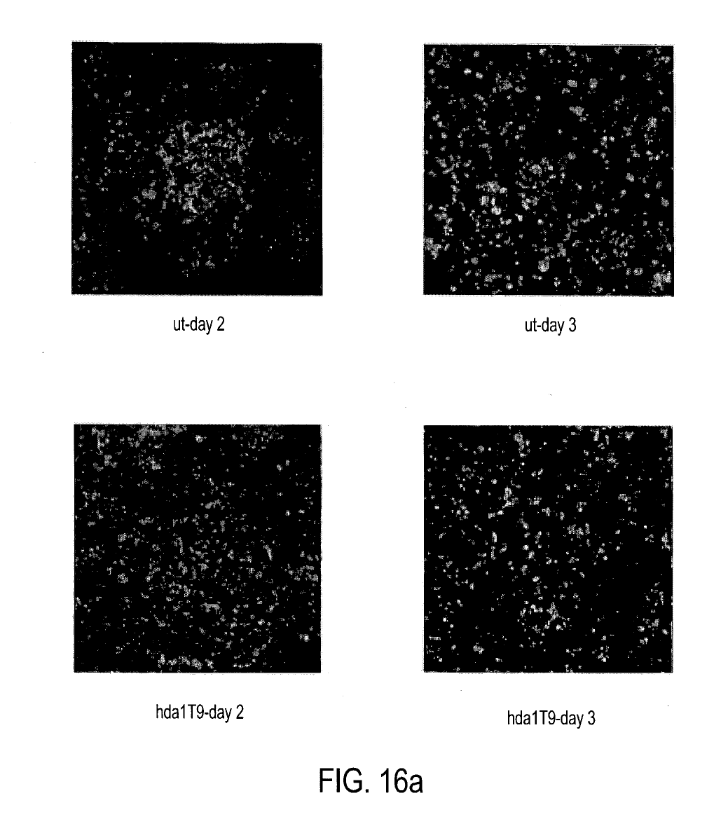

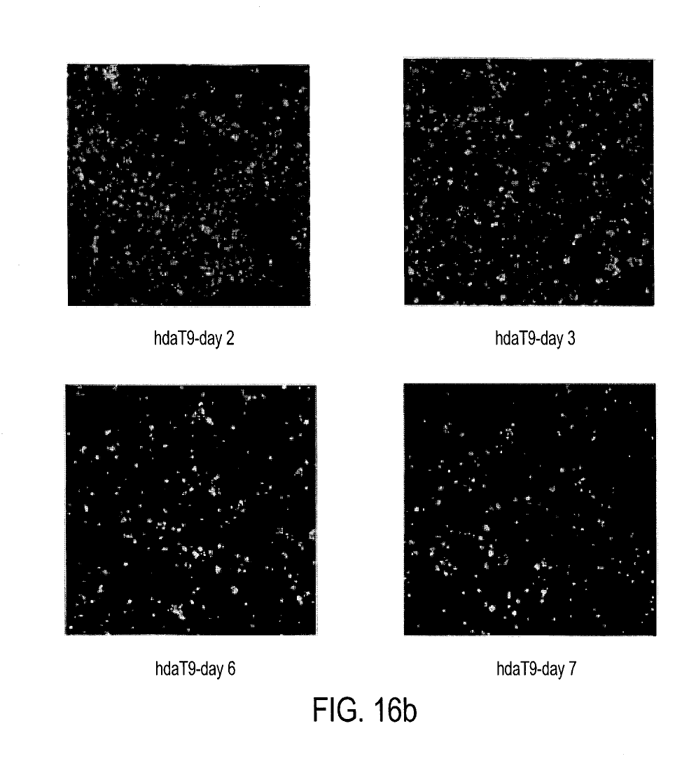

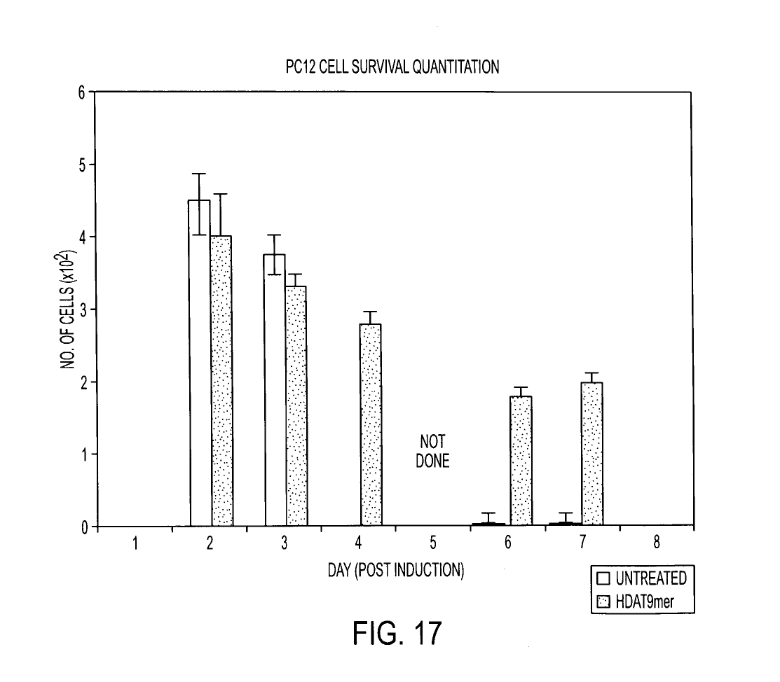

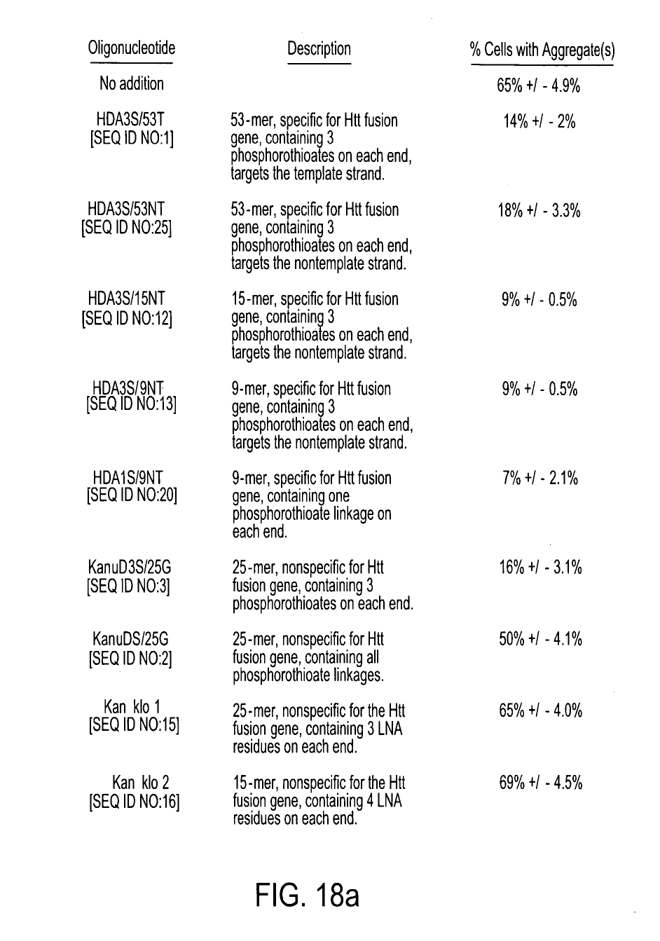

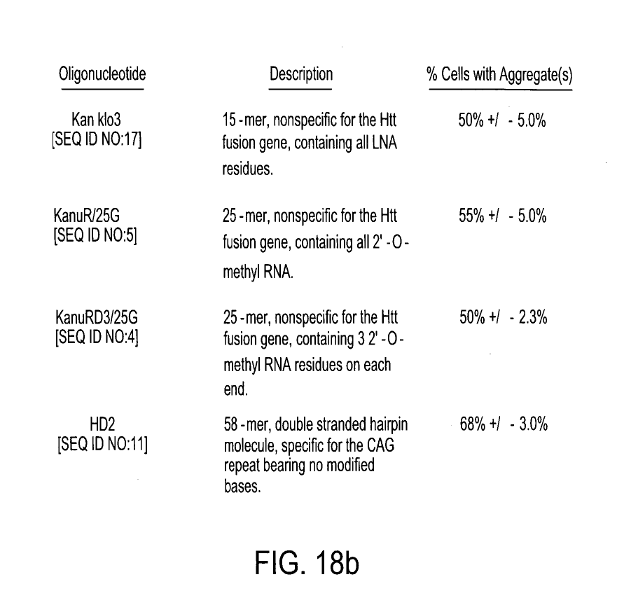

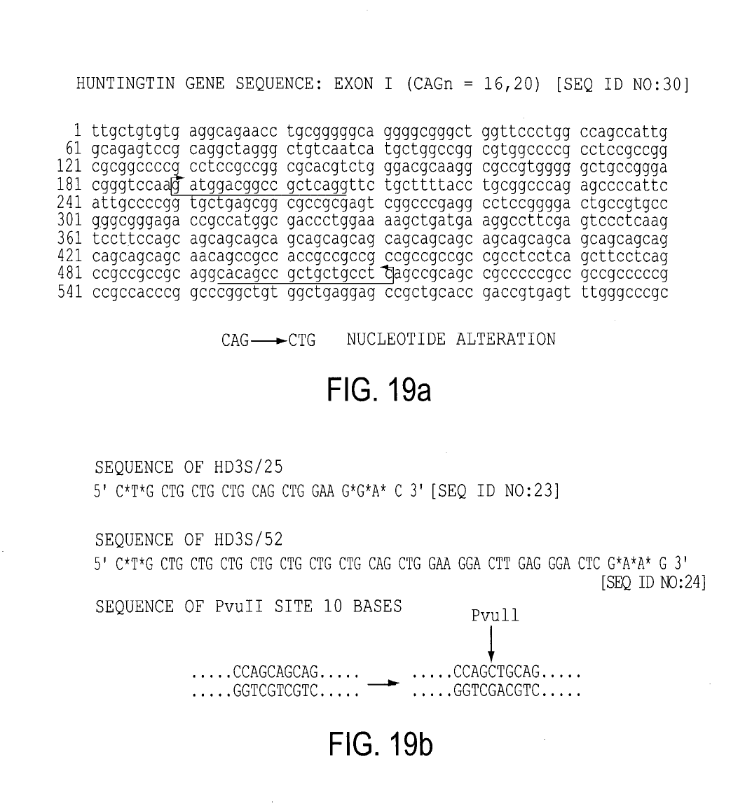

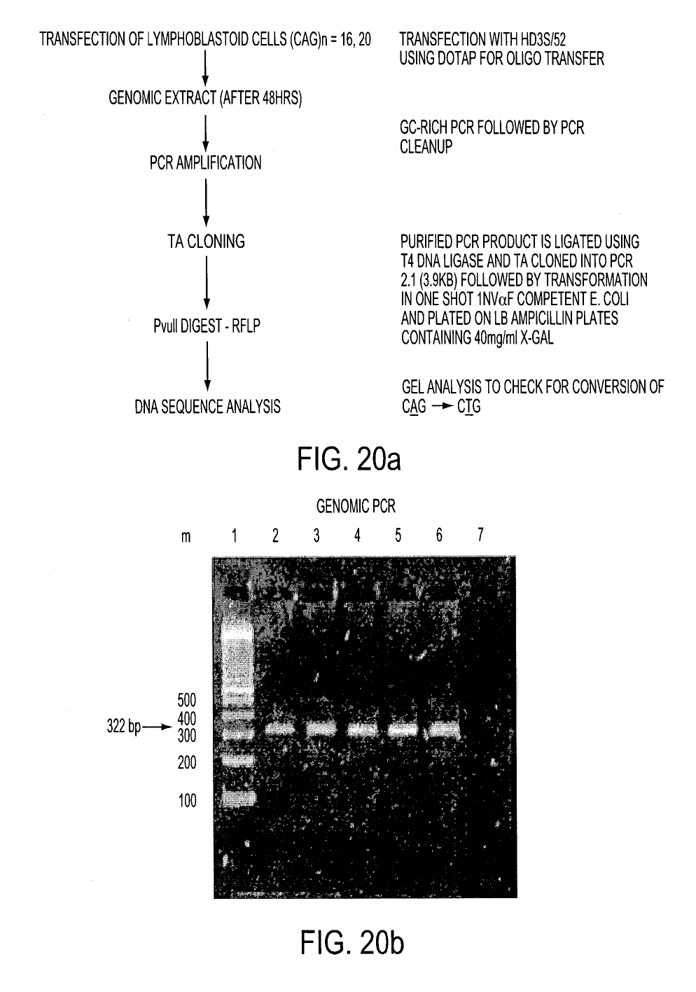

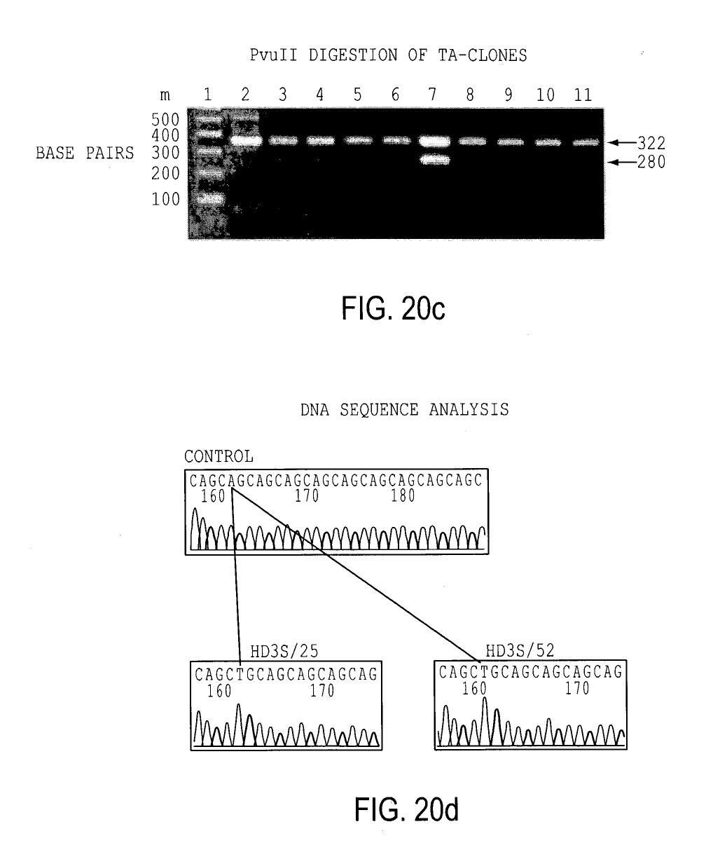

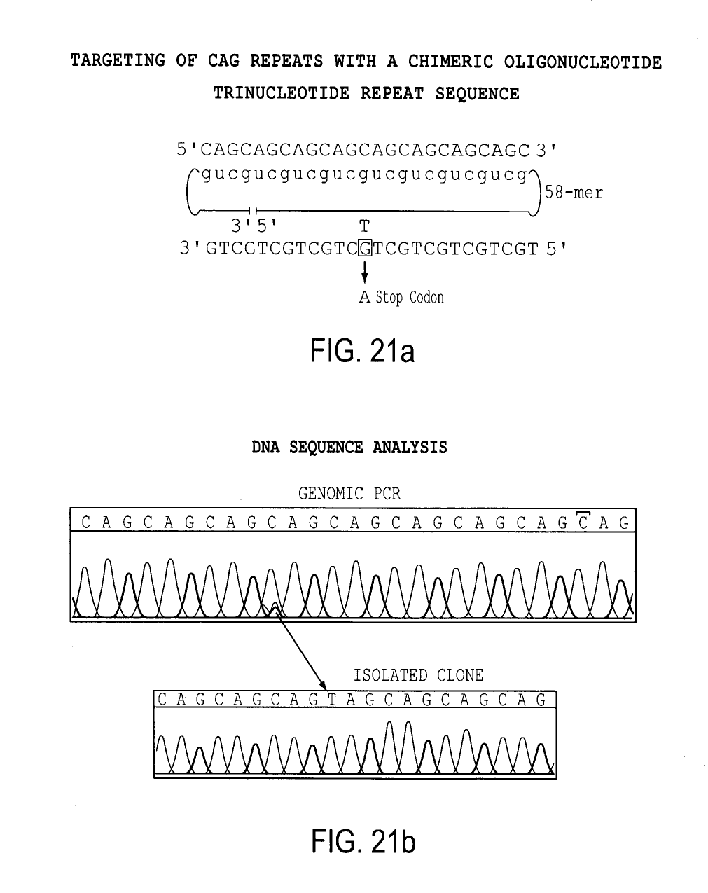

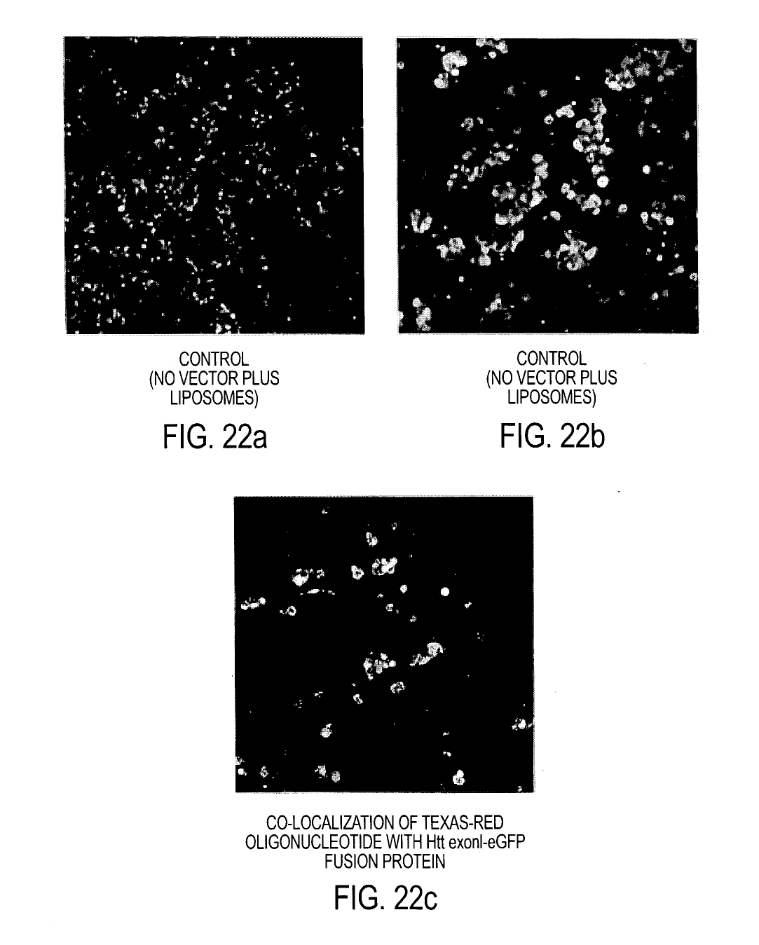

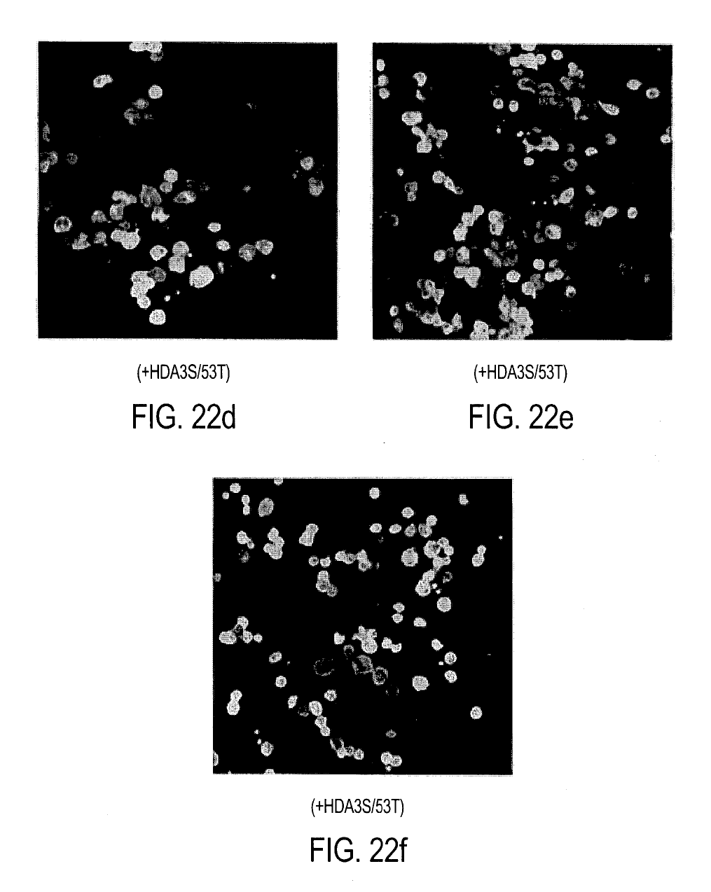

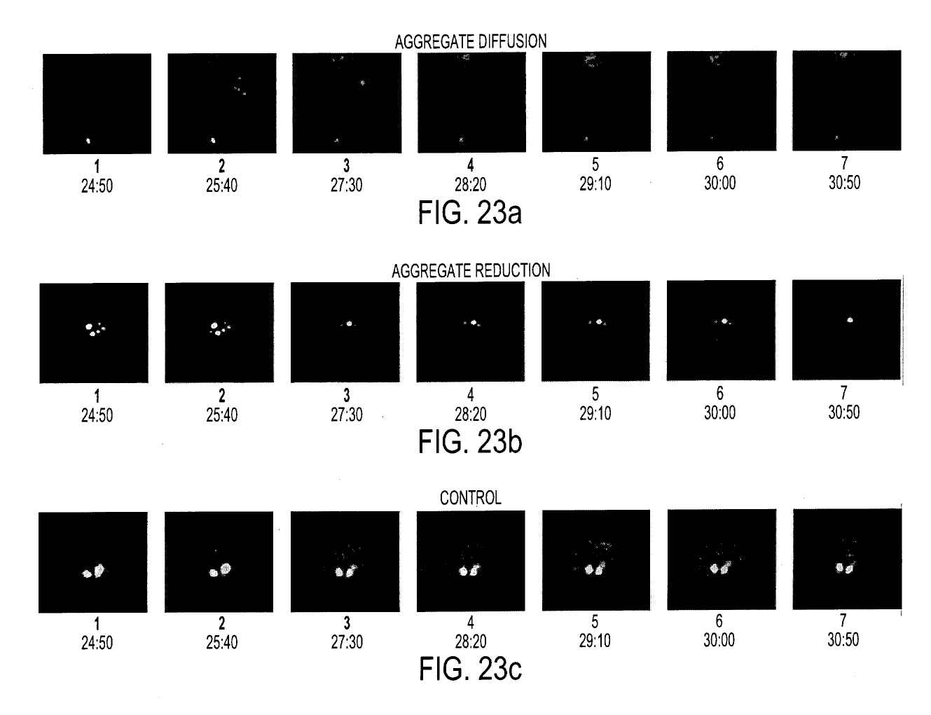

When the HD allele of a patient has n between 30 and 40, the patient is considered predisposed for HD. When the HD allele of a patient has n of about 40-50, the patient's disease is at an intermediate level of severity. When the HD allele of a patient has n of 55 or greater, the patient's disease is at a serious level. When the HD allele of a patient has n of greater than 120, the patient's condition is very serious. FIG. 2(b) displays both stands of HD gene exon 1.

FIG. 2(b) shows the DNA sequence of another wild-type (“WT”) allele of human HD exon 1, both strands. This sequence has an accession number of L27350. Note that this allele has 23 CAG or CAA codons in the CAG/CAA stretch encoding the poly Q stretch of Huntingtin protein.

FIG. 3 is a flow chart showing the sequence and structure of a HD1 chimera and an experimental strategy of using a HD1 RNA/DNA chimera to convert a CAG in the HD gene that encodes Huntingtin's protein's poly Q tract to CTG.

FIG. 4 shows the sequence of the target sequence to be converted by a HD1 chimera in the HD gene; the sequence of the allele specific polymerase chain reaction (“ASPCR”) rightward primer and the converted sequence.

FIG. 5 depicts an example of an ASPCR experiment using a HD1 chimera to correct the HD gene in 293 cells (see Example 1).

FIG. 6 shows an RNA/DNA chimeric oligonucleotide (the DNA are in upper case and the RNA in lower case) for the conversion of a CAG in the HD gene to a TAG (FIG. 6a); and the result of an exemplary experiment (FIG. 6b).

FIG. 7 is a flow chart displaying an experimental strategy for work relating to Huntingtin exon 1-GFP (green fluorescent protein) aggregation.

FIG. 8 shows the result of a representative experiment using HDA3T/53 to effect targeted alteration of the HD gene and to inhibit Huntingtin-GFP (green fluorescent protein) protein aggregation (see Example 2).

FIG. 9 is a Table with the results, in terms of number of Huntingtin-GFP protein aggregates, of an exemplary experiment using HDA3T/53 to effect targeted alteration of the HD gene (see Example 2).

FIG. 10 illustrates the concept of the use of an LNA trapper in repair of the Huntington disease gene, an example being HDA3T/53; see Table IIIa and Examples 2 and 4.

FIG. 11 shows an example of an experiment using non-specific oligonucleotides to inhibit Huntingtin-GFP protein aggregation (see Example 3).

FIG. 12 shows another example of an experiment using non-specific oligonucleotides to inhibit Huntingtin-GFP protein aggregation (see Example 3).

FIG. 13 shows the sequence of two representative modified single stranded oligonucleotides, HD3T/25 and HD3T/52, designed for targeted alteration of the HD gene. Each of these two oligonucleotides comprise three phosphorothioate linkages at each terminus.

FIG. 14 illustrates a DNA sequence analysis of altered HD gene sequence. The alteration is produced by a targeted alteration by the modified single stranded oligonucleotides shown in FIG. 13.

FIG. 15 shows an example of experiments using specific and non-specific oligonucleotides to inhibit Huntingtin-GFP protein aggregation (see Example 6).

FIG. 16 shows an example of experiments using specific oligonucleotides to inhibit Huntingtin-GFP protein aggregation (see Example 6).

hda1T9=HDA1T9mer. hdaT9=HDAT9mer.

FIG. 17 shows a PC12 cell survival quantitation graph.

FIG. 18 tabulates the percentage of cells having huntingtin (htt) aggregates after treatment with the indicated oligonucleotide, according to the present invention.

FIG. 19 shows the molecular strategy for targeted gene alteration of the htt gene, according to the present invention.

FIG. 20A shows the protocol for effecting and assessing gene alteration in the htt gene.

FIG. 20B shows genomic PCR results.

FIG. 20C shows RFLP analysis of cloned PCR products.

FIG. 20D shows sequence analysis, indicating targeted alteration of sequence in the htt gene using either HD3S/25 or HD3S/52.

FIG. 21A shows the sequence and molecular strategy for using a chimeric, double-stranded, oligonucleotide to effect gene alteration in the htt gene.

FIG. 21B presents sequence analysis, indicating a targeted change in the htt gene sequence.

FIGS. 22A-22C show fluorescent micrographs of various control experiments.

FIGS. 22D-22F show decrease in aggregation upon treatment with HDA3S/53T oligonucleotide.

FIGS. 23A-23C show diffusion of aggregates, reduction in the number of aggregates, and control, respectively.

### DETAILED DESCRIPTION OF THE INVENTION

The present invention is based upon several surprising discoveries.

We have discovered that oligonucleotides can be designed to target sequence alterations to the triplet repeat region of the Huntington's disease gene. Our early attempts to use oligonucleotides consisting of the complementary sequence to the entire CAG repeat region failed to direct detectable single-base nucleotide alteration; we have since discovered that designing the oligonucleotide so that the 5′ end of the oligonucleotide hybridizes in the unique region of the first exon, with only a part of the oligonucleotide being complementary to the CAG repeat region, permits targeted alteration.

We have also discovered that such targeted alterations reduce aggregations of the huntingtin protein (htt) in cells, and increase cell survival, effects that, although desired, could not have been predicted.

And in a surprising outgrowth of our studies using targeting oligonucleotides, we have discovered that certain oligonucleotides that are incapable of directing sequence alteration are, nonetheless, capable of reducing cellular aggregation of htt.

Assays for Measuring Protein Aggregation

In designing or screening oligonucleotides for use in the methods of the present invention, any suitable assay can be used to measure huntingtin protein (htt) aggregation, and thus measure the efficacy of oligonucleotide therapeutics of the present invention.

For example, the HD gene, or portion thereof, can be fused to another gene, or a portion thereof, which encodes a marker protein or polypeptide, or a portion thereof, or which encodes an epitope tag, such as a MYC tag. In such case, an antibody directed to the marker protein or tag can be used to detect the fusion protein comprising huntingtin. That antibody can usefully be tagged a fluorophore, such as fluorescein isothiocyanate (FITC), or another label. The antibody can also be stained by a secondary antibody that is tagged with a fluorophore, such as FITC, or another label. The aggregates can then be visualized by, for example, fluorescent microscopy.

The fusion partner can, for example, be the green fluorescent protein (GFP) gene, or a portion or derivative thereof. The huntingtin-GFP fusion protein aggregation may be monitored by monitoring the fluorescence of GFP, by, for example, fluorescent microscopy. In another alternative, the aggregation may be monitored by monitoring cell survival.

The huntingtin protein, or a portion thereof, whether part of a fusion protein or not, may also be detected by antibodies with specificity for the huntingtin moiety. The antibody to the Huntingtin protein can be tagged with a fluorophore, such as FITC, or another label. The antibody to the Huntingtin protein can also be stained by a secondary antibody that is tagged with FITC, or another label. The aggregates can then be visualized by, for example, fluorescent microscopy.

Oligonucleotides for Targeted Alteration of the Genetic Sequence of the Huntington's Disease Gene

In a first aspect, the invention provides oligonucleotide compositions—and in a related aspect, methods using the oligonucleotide compositions—for treating Huntington's disease by targeted gene alteration. Targeted alteration in one or both genomic copies of the HD gene interferes with one or more of the further expansion, continued expression, and/or aggregation of the huntingtin expanded polyglutamine tract.

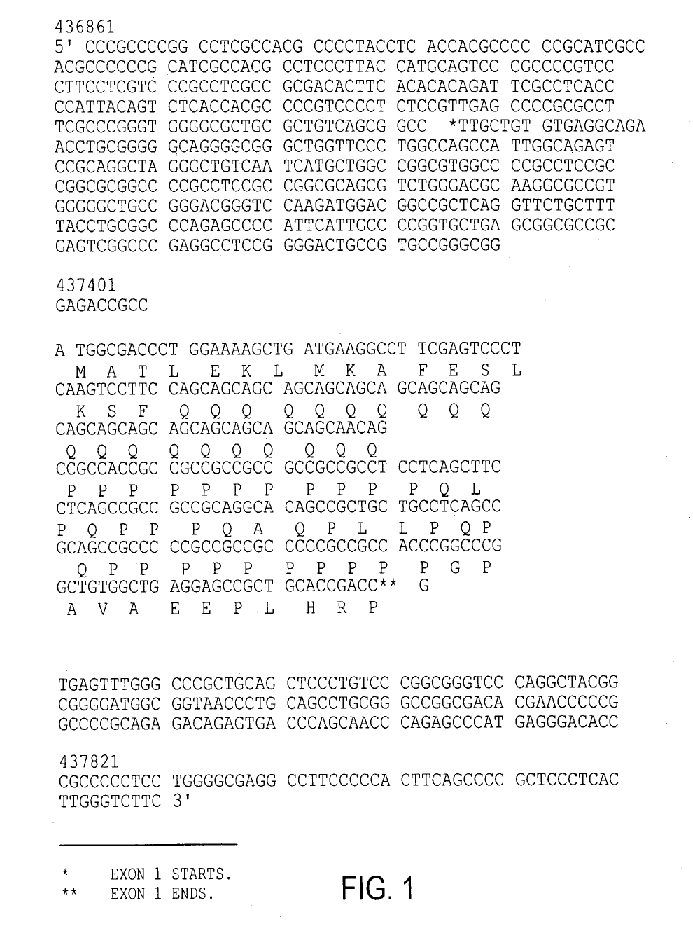

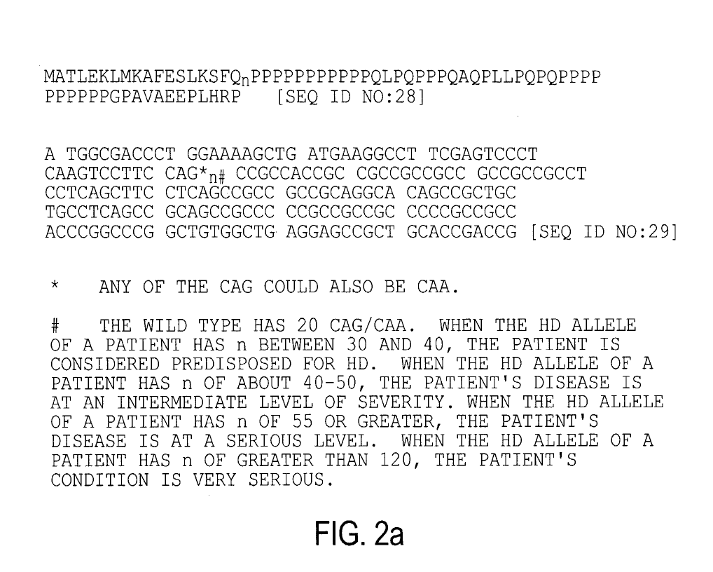

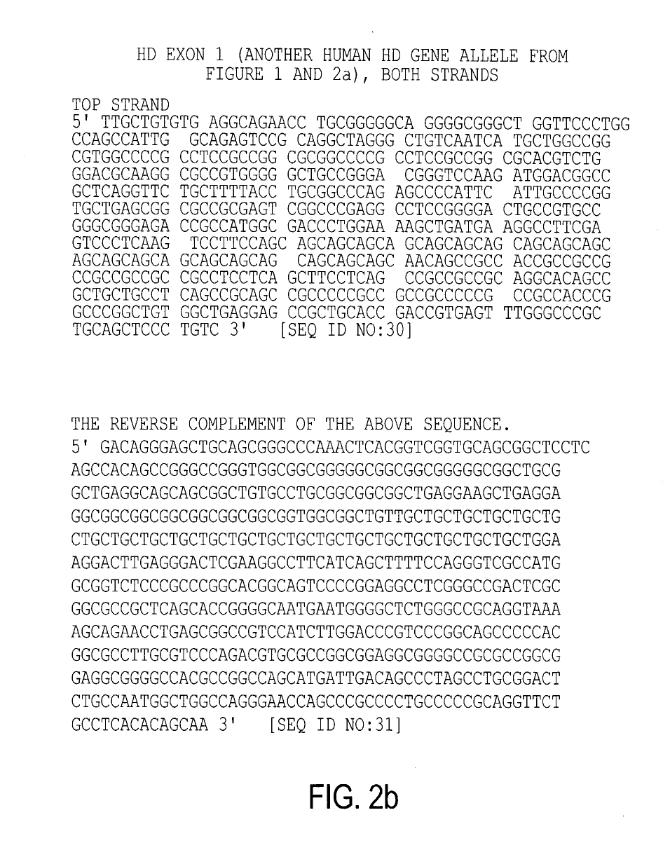

FIG. 1 shows the chromosomal DNA sequence of exon 1 of a wild-type allele of the human HD gene, as well as the chromosomal DNA sequences just upstream and just downstream of exon 1 of the human HD gene. FIG. 1 shows one allele of this portion of the HD locus. Allelic variations of the human HD gene exist; several wild type (WT), as well as mutant, alleles of the human HD gene exist. FIG. 1 also shows the amino acid sequence encoded by exon 1 of a human HD gene. FIG. 2a shows that the poly Q stretch can be expanded in exon 1 of the Huntingtin protein, such that number of Q in that stretch is one or greater. FIG. 2a also shows the codon specifying glutamine in exon 1 of a wild-type HD gene can be expanded, such that the number of codons specifying glutamine in that stretch of exon 1 of the HD gene can be one or greater. The codon that specifies the glutamine may be any codon capable of specifying glutamine (i.e., CAA and CAG). In a model system, the poly Q stretch can be encoded by approximately alternating CAA and CAG codons.

The Huntingtin protein aggregates can be formed by portions of the Huntingtin protein that comprises Huntingtin protein exon 1, or a portion thereof.

In the methods of this aspect of the present invention, oligonucleotide molecules that alter the genomic HD gene sequence—such as triplexing oligonucleotides, chimeric RNA/DNA double stranded double hairpin oligonucleotides or modified single stranded oligonucleotides—reduce the genetic instability and expansion of trinucleotide repeats, especially those associated with HD, by interrupting the triplet region encoding repetitive residues of glutamine (CAG or CAA); this reduces the propensity of the Huntingtin protein to form intracellular aggregates.

Oligonucleotides designed for use in the alteration of genetic information—whether triplexing, double-hairpin chimeric, or modified single-stranded—are significantly different from oligonucleotides designed for antisense approaches.

For example, antisense oligonucleotides are perfectly complementary to and bind an mRNA strand in order to modify expression of a targeted mRNA. As a consequence, they are unable to produce a gene conversion event by either mutagenesis or repair of a defect in the chromosomal DNA of a host genome. The backbone chemical composition used in most oligonucleotides designed for use in antisense approaches additionally renders them, in many instances, inactive as substrates for homologous pairing or mismatch repair enzymes. Furthermore, antisense oligonucleotides must be complementary to the mRNA and, therefore, will not be complementary to the other DNA strand or to genomic sequences that span the junction between intron sequence and exon sequence. Finally, the high concentrations of oligonucleotide required for antisense applications can be toxic with some types of nucleotide modifications.

Oligonucleotides of this invention that function to alter the HD gene sequence (hereinafter, “HD-specific oligonucleotides”, “oligonucleotides specific for HD”, or linguistic equivalents thereof) hybridize to at least one strand of an allele of the HD gene exon 1. FIG. 3 shows both strands of an allele of part of the human HD gene exon 1.

An oligonucleotide of this aspect of the invention can be of any sequence or length, provided that the oligonucleotide that is specific to the HD gene can hybridize to an allele of the HD gene, preferably to exon 1 of the HD gene or to exon 1 and to either the sequence upstream or downstream of exon 1, and have at least one mismatch with the HD gene so that the oligonucleotide that is specific to the HD gene can effect a HD gene alteration event. Such gene alteration events include converting a CAG to TAG, converting a CAG or CAA to any codon that specifies an amino acid other than a glutamine, and frameshift changes. The alteration caused by an oligonucleotide of this aspect of the invention may comprise an insertion, deletion, substitution, as well as any combination of these.

In one embodiment of this aspect of the invention, an oligonucleotide that is specific to the HD gene comprises a nucleic acid having a sequence of (or having a sequence complementary to that of) exon 1 of an allele of the HD gene that is just upstream or just downstream of the CAG/CAA repeats encoding the poly Q stretch (the poly Q stretch starts at amino acid residue 18 of the Huntingtin protein). Such oligonucleotide can further comprise nucleotide(s) specifying codons which encode or are complementary to codons which encode the amino acid glutamine of any number more than one, preferably more than twenty. The oligonucleotide sequence can be of any length but is preferably 300 nucleotides or shorter in length. In a preferred embodiment, the oligonucleotide comprises nucleic acid that encodes or is complementary to the DNA sequence of an allele of the HD gene that is 5′ or 3′ to the CAG/CAA repeats encoding the poly Q stretch and preferably extends into the region that encodes or is complementary to at least one of the CAG/CAA repeats.

In a preferred embodiment, an oligonucleotide that is specific to the HD gene comprises at least one mismatch with respect to the genetic sequence of an allele of the Huntington's disease gene to be altered. In a more preferred embodiment, that mismatch is to a CAG or CAA codon (any CAG/CAA codon) of the HD gene, preferably one encoding a Q in the poly Q stretch.

In the case where the gene alteration event is a frameshift, it is preferred that the initial insert or deletion resulting in the oligonucleotide mismatching the target is directed to a CAG/CAA codon or its complement that is near the 5′ end of an allele of the HD gene exon 1, i.e., closer to the ATG that specifies the initiation methionine. In another preferred embodiment, in the case where a frameshift is desired, the mismatch can be to one or more nucleotides 5′ of the CAG/CAA repeats.

In yet another preferred embodiment, an oligonucleotide that is specific to the HD gene hybridizes to either strand of an allele of the HD gene. In a more preferred embodiment, an oligonucleotide that is specific to the HD gene hybridizes to the non-transcribed strand of an allele of the HD gene.

In one embodiment, an HD-specific oligonucleotide of this aspect of the invention is a triplex-forming oligonucleotide. In another embodiment, an HD-specific oligonucleotide of this aspect of the invention is a chimeric RNA/DNA double stranded hairpin oligonucleotide (illustrations of which are provided at Table IIIB, below).

In another embodiment, presently more preferred, an HD-specific oligonucleotide of this invention is a modified single stranded oligonucleotide.

The single-stranded oligonucleotide has an internally unduplexed domain of at least 8 contiguous deoxyribonucleotides (“DNA domain”). The DNA domain is fully complementary in sequence to the sequence of a first strand of the genomic HD gene target, but for one or more mismatches as between the sequences of the oligonucleotide DNA domain and its complement on the target nucleic acid first strand. Each of the mismatches is positioned, preferably, at least 8 nucleotides from the oligonucleotide's 5′ and 3′ termini.

Furthermore, the oligonucleotide will typically have at least one terminal modification selected from the group consisting of: at least one terminal locked nucleic acid (LNA), at least one terminal 2′-O—Me base analog, and at least three terminal phosphorothioate linkages.

An example of a modified single-stranded oligonucleotide of this aspect of the invention is HDA3T/53. See Table IIIa and Examples 2 and 4, below. The modification of these oligonucleotides is described below.

The frequency of gene alteration by unmodified oligonucleotides is low. Without intending to be bound by theory, the low efficiency of gene alteration obtained using unmodified DNA oligonucleotides is believed to be largely the result of degradation by nucleases present in the reaction mixture or the target cell. Nucleic acid analogs have been developed that increase the nuclease resistance of oligonucleotides that contain them, including, e.g., nucleotides containing phosphorothioate linkages or 2′-O-methyl analogs present at least on the 3′ end of the oligonucleotide.

The efficiency of gene alteration is increased, in single-stranded oligonucleotides having internal complementary sequence to a target, when the oligonucleotide comprises phosphorothioate modified bases as compared to 2′-O-methyl modifications.

Similarly, locked nucleic acid (LNA) analogs provide modifications which allow for increased efficiency of alteration of a gene. LNAs and LNA analogues and derivatives, such as xylo-LNAs and L-ribo-LNAs, are described in international patent publications WO 99/14226, WO 00/56748, and WO 00/66604, the disclosures of which are incorporated herein by reference in their entireties.

Oligonucleotides comprising 2′-O-methyl or LNA analogs are a mixed DNA/RNA polymer. These oligonucleotides are, however, single-stranded and are not designed to form a stable internal duplex structure within the oligonucleotide, as are linear double-stranded, double-hairpin, chimeric HD-specific oligonucleotides.

In a preferred embodiment, a single stranded oligonucleotide of this invention comprises one or more chemical modifications selected from the group consisting of an O-methyl modification, an LNA modification, including LNA derivatives and analogs, two or more phosphorothioate linkages on one or more termini, and a combination of any two or more of these modifications. In a more preferred embodiment, the single stranded oligonucleotide comprises two or more phosphorothioate linkages on at least the 3′ terminus. In an even more preferred embodiment, the single stranded oligonucleotide comprises two or more phosphorothioate linkages on both termini.

In another preferred embodiment, a single stranded oligonucleotide of this invention, which has a DNA domain, the DNA domain having at least one mismatch with respect to the genetic sequence of the Huntington's disease gene to be altered, further comprises a 2′-O-methyl analog.

In yet another preferred embodiment, the single stranded oligonucleotide comprises an LNA nucleotide, including an LNA derivative or analog. In yet another preferred embodiment, the single stranded oligonucleotide comprises a combination of at least two modifications selected from the group consisting of a phosphorothioate linkage, a 2′-O-methyl analog, a locked nucleotide analog and a ribonucleotide. In yet another preferred embodiment, the single stranded oligonucleotide comprises unmodified ribonucleotide.

For the oligonucleotides of this aspect of the invention, the optimum length, optimum sequence, optimum position of the mismatched base or bases, optimum chemical modification or modifications, and optimum strand targeted, can be easily determined for a particular gene alteration event by comparing to a control, such as an oligonucleotide perfectly complementary to one of the HD alleles, or an oligonucleotide lacking terminal and internal modifications.

The modified single stranded oligonucleotides that are specific for the HD gene include, for each correcting change, oligonucleotides of length 4, 9, 15, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120, with further single-nucleotide additions up to about 300 nucleotides. Moreover, the single stranded oligonucleotides of the invention do not require a symmetrical extension on either side of the central DNA domain. Similarly, the modified single stranded oligonucleotides of the invention contain phosphorothioate linkages, 2′-O-methyl analogs or LNAs or any combination of these modifications.

Oligonucleotides of this aspect of the invention may be altered with any combination of additional LNAs, phosphorothioate linkages or 2′-O-methyl analogs to maximize conversion efficiency. For oligonucleotides that are longer than about 17 to about 25 bases in length, internal as well as terminal region segments of the oligonucleotide may be altered. Alternatively, simple fold-back structures at each end of a oligonucleotide or appended end groups may be used in addition to a modified backbone to increase efficiency of targeted alteration.

The oligonucleotides described herein preferably contain more than one of the aforementioned modifications (collectively referred to as “backbone modifications”) at each end. In some embodiments, the backbone modifications are adjacent to one another. However, the optimal number and placement of backbone modifications for any individual oligonucleotide will vary with the length of the oligonucleotide and the particular type of backbone modification(s) that are used. If constructs of identical sequence having phosphorothioate linkages are compared, 2, 3, 4, 5, or 6 phosphorothioate linkages at each end are preferred. If constructs of identical sequence having 2′-O-methyl or LNA base analogs are compared, 1, 2, 3 or 4 analogs are preferred. Some oligonucleotides comprising LNA base analogs do not function for altering target DNA.

The optimal number and type of backbone modifications for any particular oligonucleotide useful for altering target DNA may be determined empirically by comparing the alteration efficiency of the oligonucleotide comprising any combination of the modifications to a control molecule of comparable sequence using any of the assays described herein.

Analogously, the optimal position(s) for oligonucleotide modifications for a maximally efficient altering oligonucleotide can be determined by testing the various modifications as compared to control molecule of comparable sequence in one of the assays disclosed herein.

The oligonucleotides of this aspect of the invention may target either strand of the genomic htt locus, and can include any sequence drawn from any component of the genome including, for example, intron and exon sequences. Presently preferred embodiments bind to the non-transcribed strand of a genomic DNA duplex.

As described above, the modified single stranded, HD-specific, oligonucleotides of the present invention for alteration of a base of the HD gene are preferably about 4 to about 300 nucleotides in length, more preferably from about 9 to 74, more preferably about 9 to 53 nucleotides in length. Most preferably, however, these oligonucleotides are at least about 25 bases in length, unless there are self-dimerization structures within the oligonucleotide.

If the oligonucleotide has self-dimerization structures, lengths longer than 35 bases are preferred. Oligonucleotides with modified ends both shorter and longer than certain of the exemplified, modified oligonucleotides herein function as gene repair or gene knockout agents and are within the scope of the present invention.

Once an oligomer is chosen, it can be tested for its tendency to self-dimerize, since self-dimerization may result in reduced efficiency of alteration of genetic information. Checking for self-dimerization tendency can be accomplished manually or, more preferably, by using a software program. One such program is Oligo Analyzer 2.0, available through Integrated DNA Technologies (Coralville, Iowa 52241) (http://www.idtdna.com); this program is available for use on the world wide web at

http://www.idtdna.com/program/oligoanalyzer/oligoanalyzer.asp.

For each oligonucleotide sequence input into the program, Oligo Analyzer 2.0 reports possible self-dimerized duplex forms, which are usually only partially duplexed, along with the free energy change associated with such self-dimerization. Delta G-values that are negative and large in magnitude, indicating strong self-dimerization potential, are automatically flagged by the software as “bad”. Another software program that analyzes oligomers for pair dimer formation is Primer Select from DNASTAR, Inc., 1228 S. Park St., Madison, Wis. 53715, Phone: (608) 258-7420 (http://www.dnastar.com/products/PrimerSelect.html). If the sequence is subject to significant self-dimerization, the addition of further sequence flanking the “repair” nucleotide can improve gene correction frequency.

Generally, the modified single stranded oligonucleotides of this aspect of the present invention are identical in sequence to one strand of the HD target DNA, which can be either strand of the target DNA, with the exception of one or more targeted bases positioned within the DNA domain of the oligonucleotide, typically greater than or equal to 8 nucleotides from each of the oligonucleotide's termini. In a preferred embodiment, the oligonucleotides of the invention are complementary to the non-transcribed strand of a duplex.

The modified single stranded oligonucleotides of the present invention that are specific for the HD gene can include more than a single base change. In an oligonucleotide that is about a 70-mer, with at least one modified residue incorporated on the ends, as disclosed herein, multiple bases can be simultaneously targeted for change. The target bases may be up to 27 nucleotides apart and may not be changed together in all cases. The farther apart the two target bases are, the less frequent the simultaneous change. Thus, oligonucleotides of the invention may be used to repair or alter multiple bases rather than just one single base.

This invention thus provides, in one embodiment, an oligonucleotide, useful for altering at least one glutamine codon within the poly-Q stretch in exon-1 of the HD gene, which oligonucleotide is preferably a chimeric RNA/DNA oligonucleotide, more preferably a modified single stranded oligonucleotide, and which can convert a CAA or a CAG in the polyQ track of HD gene to any other codon that does not specify glutamine or to a stop codon. This invention also provides an oligonucleotide that can alter the HD gene by causing a frameshift mutation within the poly-Q track of HD or just preceding the poly-Q track of HD. All these genetic alterations lead to inhibition of Huntingtin protein aggregation and cause disaggregation of Huntingtin protein aggregates.

Methods of Using an Oligonucleotide for Targeted Alteration of the Genetic Sequence of the Huntington's Disease Gene

This invention also provides methods of using the HD-specific, gene-altering oligonucleotides to prevent (for example, prior to the onset of the disease or the appearance of protein aggregates), or treat Huntington's disease (for example, after the onset of the disease or the appearance of protein aggregates).

The method comprises administering to a subject an effective amount of an HD-specific oligonucleotide as above-described.

The treating oligonucleotide preferably is a single-stranded oligonucleotide lacking a double-stranded, double-hairpin structure, and having one or more chemical modifications, preferably selected from the group consisting of: an O-methyl modification, an LNA modification, including LNA derivatives and analogs, one or more phosphorothioate linkages, preferably on one or more termini but permissible throughout, and a combination of any two or more of these modifications. The oligonucleotide is designed to alter the HD gene sequence.

Administration of oligonucleotides decreases aggregation of huntingtin in cells; without intending to be bound by theory, it is believed that the decrease is due to a decreased rate of formation, and/or a reduced rate of further triplet expansion.

The oligonucleotide is administered to a subject in need thereof at or above therapeutically effective concentrations, which may result in protein disaggregation or reduced rate of formation of protein aggregates.

Although a preferred HD-specific, sequence-altering oligonucleotide is a single-stranded chemically modified oligonucleotide as above-described, the methods of this aspect of the present invention may also be practiced using a linear double-stranded, double-hairpin-containing chimeric oligonucleotide and a triplexing gene-altering oligonucleotide, also as above-described.

Route of Administration

The oligonucleotides described herein can be introduced into cells by any suitable means. The modified oligonucleotides may be used alone. Suitable means include the use of polycations, cationic lipids, liposomes, polyethylenimine (PEI), electroporation, biolistics, microinjection and other methods known in the art to facilitate cellular uptake. Other suitable means include direct injection into the spinal fluid, the region of the caudate nucleus or the putamen or by administration into cells via injection into the nucleus, biolistic bombardment, electroporation, liposome transfer and calcium phosphate precipitation. In a preferred method of cellular administration, the administration is performed with a liposomal transfer compound, e.g., DOTAP (Boehringer-Mannheim) or an equivalent such as lipofectin.

In other instances, targeted genomic alteration, including repair or mutagenesis, may take place in vivo following direct administration of the oligonucleotides of this invention to a subject.

Effective amounts of the oligonucleotides of this invention are preferably administered to the subject in the form of an injectable composition. The composition is preferably administered parenterally, meaning intravenously, intraarterially, intrathecally, interstitially or intracavitarilly.

Pharmaceutical compositions of this invention can be administered to mammals including humans in a manner similar to other diagnostic or therapeutic agents. An oligonucleotide of short length, such as an oligonucleotide that is about 4 to 15 nucleotides in length, can be administered as a small molecule. A small molecule such as an oligonucleotide that is about 4 to about 15 nucleotides in length may be more able to cross the blood/brain barrier.

The dosage to be administered, and the mode of administration will depend on a variety of factors including age, weight, sex, condition of the subject and genetic factors, and will ultimately be decided by medical personnel subsequent to experimental determinations of varying dosage as described herein. In general, dosage required for prophylaxis or correction and therapeutic efficacy will range from about 0.001 to 50,000 μg/kg, preferably between 1 to 250 μg/kg of host cell or body mass, and most preferably at a concentration of between 30 and 60 micromolar.

Formulation

A purified oligonucleotide composition comprising an oligonucleotide of the present invention may be formulated in accordance with routine procedures as a pharmaceutical composition adapted for bathing cells in culture, for microinjection into cells in culture, and for intravenous or local administration, or any other form of administration, to human beings or animals. Typically, compositions for cellular administration or for intravenous or local administration into animals, including humans, are solutions in sterile isotonic aqueous buffer. Where necessary, the composition may also include a solubilizing agent and a local anaesthetic such as lignocaine to ease pain at the site of the injection. Generally, the ingredients will be supplied either separately or mixed together in unit dosage form, for example, as a dry, lyophilized powder or water-free concentrate. The composition may be stored in a hermetically sealed container such as an ampule or sachette indicating the quantity of active agent in activity units. Where the composition is administered by infusion, it can be dispensed with an infusion bottle containing sterile pharmaceutical grade “water for injection” or saline. Where the composition is to be administered by injection, an ampule of sterile water for injection or saline may be provided so that the ingredients may be mixed prior to administration.

Pharmaceutical compositions of this invention comprise the oligonucleotides of the present invention and pharmaceutically acceptable salts thereof, with any pharmaceutically acceptable ingredient, excipient, carrier, adjuvant or vehicle.

The oligonucleotides of this invention may be administered in a pharmaceutically effective, prophylactically effective or therapeutically effective amount, which is an amount sufficient to produce a detectable, preferably medically beneficial, effect on a subject at risk or afflicted with HD, which effects may include disaggregation of huntingtin protein aggregates or inhibition of the formation of huntingtin protein aggregates.

Subjects

Effective amounts of the oligonucleotides of this invention (chimeric RNA/DNA oligonucleotides and modified single stranded oligonucleotides that are specific for the HD gene and that can alter the HD gene sequence), can be administered for treatment or prophylaxis to any mammalian subject suffering or about to suffer HD. Preferably, the subject is a primate, more preferably a higher primate, most preferably a human.

HD-Nonspecific Oligonucleotide Compositions and Methods for Disaggregation of Huntingtin Aggregations

To our great surprise, we discovered as a byproduct of the targeted gene alteration experiments reported in the Examples below that certain of the control oligonucleotides, which are incapable of effecting gene alteration, are nonetheless effective at disaggregating huntingtin aggregates. Thus, in another aspect, the present invention provides methods for identifying such oligonucleotides (hereinafter, “HD-nonspecific oligonucleotides”, or linguistic variants thereof), compositions comprising such HD-nonspecific oligonucleotides, and methods of treating Huntington's disease using such compositions.

The oligonucleotides used in the compositions and methods of this aspect of the present invention can be as short as about 4 nucleotides in length, and as long as about 25 nucleotides in length, and thus can be 4, 5, 6, 7, 8, 9, 10, 11, 12, 13, 14, 15, 16, 17, 18, 19, 20, 21, 22, 23, 24, or 25 nucleotides in length, exclusive of optional terminal blocking groups.

The oligonucleotides can comprise nucleobases naturally found in nature in native 5′-3′ phosphodiester internucleoside linkage—e.g., DNA, RNA, or chimeras thereof—or can contain any or all of nucleobases not found in nature (non-native nucleobases), nonnative internucleobase bonds, or post-synthesis modifications, either throughout the length of the oligonucleotide or localized to one or more portions thereof.

For example, the oligonucleotides of this aspect of the present invention may usefully comprise altered, often nuclease-resistant, internucleoside bonds, as are typically used in antisense applications. See, e.g., Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents—Symposium No. 209, John Wiley & Son Ltd (1997) (ISBN: 0471972797), the disclosures of which are incorporated herein by reference in their entireties.

Modified oligonucleotide backbones may include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosures of which are incorporated herein by reference in their entireties.

Other modified oligonucleotide backbones useful in the oligonucleotides of the present invention include those that lack a phosphorus atom, such as backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above backbones include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, the disclosures of which are incorporated herein by reference in their entireties.

The oligonucleotides of this aspect of the present invention may also include nonnaturally occurring nucleobases, either in standard phosphodiester linkage, where the chemistry allows, or with other types of linkage not found in naturally occurring nucleic acids (as would be clear to the person skilled in the art, various nucleobases which previously have been considered nonnaturally occurring have subsequently been found in nature).

The oligonucleotides of this aspect of the present invention may thus include nucleobases such as the known purine and pyrimidine heterocycles, and also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosine, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-S-methyl-4-triazolopyridine, isocytosine, isoguanine, inosine and the “non-naturally occurring” nucleobases described in U.S. Pat. No. 5,432,272, included herein by reference in its entirety.

Locked nucleic acid (LNA) analogues may have utility, although LNA-containing oligonucleotides tested to date have proven poorly effective in disaggregating huntingtin aggregates, as further described in the Examples below.

The oligonucleotides of this aspect of the present invention may also usefully include 2′-OMe analogues; when linked to deoxyribonucleotides in 5′-3′ phosphodiester bonds, the resulting oligonucleotide is a chimera of RNA and DNA.

Differences from nucleic acid compositions found in nature—e.g., altered internucleoside linkages, nonnaturally occurring nucleobases, and post-synthetic modifications—can be present throughout the length of the oligonucleotide or can instead be localized to discrete portions thereof.

The oligonucleotides useful in this aspect of the present invention can also optionally include end-groups, at either or both of the 5′ and 3′ termini; such end-groups may usefully reduce degradation or, in addition or in the alternative, provide other functionalities.

For example, the 5′ terminus may be phosphorylated, either chemically or enzymatically, thus increasing the oligonucleotide's negative charge.

The 5′ end may, in the alternative, be modified to include a primary amine group, typically appended during solid phase synthesis through use of an amino modifying phosphoramidite, such as a β-cyanoethyl (CE) phosphoramidite (Glen Research, Inc., Sterling, Va.). The 5′ end may instead be modified to display a reactive thiol group, which can be appended during solid phase synthesis through use of a thiol modified phosphoramidite, such as (S-Trityl-6-mercaptohexyl)-(2-cyanoethyl)-(N,N-diisopropyl)-phosphoramidite (Glen Research, Inc., Sterling, Va.).

Amine and thiol-modified oligonucleotides can be readily conjugated to other moieties, such as proteins, lipids, or carbohydrates.

Among such moieties are usefully those that serve to target the oligonucleotide to the cell type of therapeutic interest.

For example, international patent publications WO 02/47730 and WO 00/37103, incorporated herein by reference in their entireties, describe compounds for intracellular delivery of therapeutic moieties to nerve cells. The targeting moieties are neurotrophins—such as NGF, BDNF, NT-3, NT-4, NT-6, and fragments thereof—that effect the targeted internalization of the compound by nerve cells of various classes. Such moieties may usefully be appended to the oligonucleotides of this aspect of the present invention in order to disrupt protein aggregations characteristic of Huntington's disease.

Other targeting moieties that may usefully be appended to the oligonucleotides of this aspect of the present invention facilitate passage across the blood brain barrier, such as the OX26 monoclonal antibody (reviewed in Pardridge, “Brain drug delivery and blood-brain barrier transport”, Drug Delivery 3:99-115 (1996), incorporated herein by reference in its entirety; see also U.S. Pat. Nos. 5,154,924 and 5,977,307, incorporated herein by reference in their entireties).

The 3′ end of the oligonucleotide of the present invention may similarly be amine or thiol modified to permit the ready conjugation of the oligonucleotide to, among others, proteins, carbohydrates, and lipids.

Other 5′ and 3′ end-modifications include, for example, fluorescent labels, that permit the monitoring of the extracellular and intracellular distribution of the oligonucleotide.

Fluorescent labels useful for end-modification are well known, and include, for example, fluorescein isothiocyanate (FITC), allophycocyanin (APC), R-phycoerythrin (PE), peridinin chlorophyll protein (PerCP), Texas Red, Cy3, Cy5, Cy7, and fluorescence resonance energy tandem fluorophores such as PerCP-Cy5.5, PE-Cy5, PE-Cy5.5, PE-Cy7, PE-Texas Red, and APC-Cy7.

Other fluorophores usefully appended to the 5′ or 3′ ends of the oligonucleotides of the present invention include, inter alia, Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits available from Molecular Probes, Inc., Eugene, Oreg., USA), BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3.5, and Cy5.5.

The oligonucleotides may also include a 3′ and/or 5′ group useful for secondary labeling or purification, such as biotin, dinitrophenyl, or digoxigenin.

When the sequence desired for the oligonucleotide is known, the oligonucleotides of the present invention can usefully be synthesized using standard solid phase chemistries appropriate to the nucleobases and linkages desired.

When the sequence desired is yet to be determined, the oligonucleotides of the present invention can usefully be synthesized combinatorially, providing oligonucleotides of all possible sequences for any desired length of oligonucleotide, from which desired sequences can thereafter be selected.

Such combinatorial methods are known in the art. In the simplest such method, all possible nucleobase monomers are used in each synthesis cycle. A disadvantage of this approach is that oligonucleotides of disparate sequence are present in admixture. Other methods permit high throughput parallel synthesis in which oligonucleotides differing in sequence are segregated. See, e.g., Cheng et al., “High throughput parallel synthesis of oligonucleotides with 1536-channel synthesizer,” Nucl. Acids Res. 30(16): _-_ (2002).

In one aspect, therefore, the invention provides a method for identifying, from a plurality of HD-nonspecific oligonucleotides differing in sequence, those oligonucleotides that are effective to disrupt aggregation of huntingtin within affected cells.

The method comprises introducing each of a plurality of oligonucleotides of disparate sequence separately into cells that have or are at risk to develop huntingtin aggregates, and identifying the oligonucleotide (or plurality of oligonucleotides) most effective at disrupting or preventing aggregation.

The oligonucleotides to be tested differ from one another in sequence. They may optionally differ additionally in composition, such as in length, in the presence, position, and number of nonnative internucleoside linkages, in the presence, position, number and chemistry of nonnative nucleobases, and in the presence, position, and number of terminal modifications.

The cells are typically cultured cells, and the oligonucleotides are thus introduced into the cells in vitro. In other embodiments, however, the cells are present within a laboratory animal, and the oligonucleotides are introduced by administration to the animal.

The cells chosen for use in this method exhibit or develop huntingtin aggregation. Several such cell lines are described in the Examples, below.

The cells can be naturally occurring, e.g. derived from a patient having or predisposed to Huntington's disease, or can be engineered. Accordingly, the protein aggregation can comprise a naturally-occurring, albeit pathologically aggregated, huntingtin aggregant, or can comprise a non-naturally occurring protein aggregant.

Among non-naturally occurring protein aggregations, fusions that comprise the protein aggregant (huntingtin), or an aggregation-competent portion thereof, and a detectable marker, are particularly useful.

Among such detectable markers, fluorescent proteins having a green fluorescent protein (GFP)-like chromophore prove particularly useful.

As used herein, “GFP-like chromophore” means an intrinsically fluorescent protein moiety comprising an 11-stranded β-barrel (β-can) with a central α-helix, the central α-helix having a conjugated π-resonance system that includes two aromatic ring systems and the bridge between them. By “intrinsically fluorescent” is meant that the GFP-like chromophore is entirely encoded by its amino acid sequence and can fluoresce without requirement for cofactor or substrate.

The GFP-like chromophore can be selected from GFP-like chromophores found in naturally occurring proteins, such as A. victoria GFP (GenBank accession number AAA27721), Renilla reniformis GFP, FP583 (GenBank accession no. AF168419) (DsRed), FP593 (AF272711), FP483 (AF168420), FP484 (AF168424), FP595 (AF246709), FP486 (AF168421), FP538 (AF168423), and FP506 (AF168422), and need include only so much of the native protein as is needed to retain the chromophore's intrinsic fluorescence. Methods for determining the minimal domain required for fluorescence are known in the art. Li et al., “Deletions of the Aequorea victoria Green Fluorescent Protein Define the Minimal Domain Required for Fluorescence,” J. Biol. Chem. 272:28545-28549 (1997).

Alternatively, the GFP-like chromophore can be selected from GFP-like chromophores modified from those found in nature. Typically, such modifications are made to improve recombinant production in heterologous expression systems (with or without change in protein sequence), to alter the excitation and/or emission spectra of the native protein, to facilitate purification, to facilitate or as a consequence of cloning, or are a fortuitous consequence of research investigation.

The methods for engineering such modified GFP-like chromophores and testing them for fluorescence activity, both alone and as part of protein fusions, are well-known in the art. Early results of these efforts are reviewed in Heim et al., Curr. Biol. 6:178-182 (1996), incorporated herein by reference in its entirety; a more recent review, with tabulation of useful mutations, is found in Palm et al., “Spectral Variants of Green Fluorescent Protein,” in Green Fluorescent Proteins, Conn (ed.), Methods Enzymol. vol. 302, pp. 378-394 (1999), incorporated herein by reference in its entirety.

A variety of such modified chromophores are now commercially available and can readily be used in the fusion proteins of the present invention.

For example, EGFP (“enhanced GFP”), Cormack et al., Gene 173:33-38 (1996); U.S. Pat. Nos. 6,090,919 and 5,804,387, is a red-shifted, human codon-optimized variant of GFP that has been engineered for brighter fluorescence, higher expression in mammalian cells, and for an excitation spectrum optimized for use in flow cytometers. EGFP can usefully contribute a GFP-like chromophore to the fusion proteins of the present invention. A variety of EGFP vectors, both plasmid and viral, are available commercially (Clontech Labs, Palo Alto, Calif., USA), including vectors for bacterial expression, vectors for N-terminal protein fusion expression, vectors for expression of C-terminal protein fusions, and for bicistronic expression.

Toward the other end of the emission spectrum, EBFP (“enhanced blue fluorescent protein”) and BFP2 contain four amino acid substitutions that shift the emission from green to blue, enhance the brightness of fluorescence and improve solubility of the protein, Heim et al., Curr. Biol. 6:178-182 (1996); Cormack et al., Gene 173:33-38 (1996). EBFP is optimized for expression in mammalian cells whereas BFP2, which retains the original jellyfish codons, can be expressed in bacteria; as is further discussed below, the host cell of production does not affect the utility of the resulting fusion protein. The GFP-like chromophores from EBFP and BFP2 can usefully be included in the fusion proteins of the present invention, and vectors containing these blue-shifted variants are available from Clontech Labs (Palo Alto, Calif., USA).

Analogously, EYFP (“enhanced yellow fluorescent protein”), also available from Clontech Labs, contains four amino acid substitutions, different from EBFP, Ormö et al., Science 273:1392-1395 (1996), that shift the emission from green to yellowish-green. Citrine, an improved yellow fluorescent protein mutant, is described in Heikal et al., Proc. Natl. Acad. Sci. USA 97:11996-12001 (2000). ECFP (“enhanced cyan fluorescent protein”) (Clontech Labs, Palo Alto, Calif., USA) contains six amino acid substitutions, one of which shifts the emission spectrum from green to cyan. Heim et al., Curr. Biol. 6:178-182 (1996); Miyawaki et al., Nature 388:882-887 (1997). The GFP-like chromophore of each of these GFP variants can usefully be included in fusion protein aggregants of the present invention.

The GFP-like chromophore can also be drawn from other modified GFPs, including those described in U.S. Pat. Nos. 6,124,128; 6,096,865; 6,090,919; 6,066,476; 6,054,321; 6,027,881; 5,968,750; 5,874,304; 5,804,387; 5,777,079; 5,741,668; and 5,625,048, the disclosures of which are incorporated herein by reference in their entireties.

Recombinant fusions of the protein aggregant (huntingtin, or an aggregation-competent fragment thereof) with a detectable marker, such as a protein comprising a GFP-like chromophore, makes it possible to detect aggregation, and disruption of aggregation, by qualitative or quantitative observation of the cellular location and local concentration of the protein aggregant.

Where the fused marker is fluorescent, e.g. a protein moiety having a GFP-like chromophore, aggregation can be observed visually, typically using a fluorescence microscope. High throughput apparatus, such as the Amersham Biosciences IN Cell Analysis System and Cellomics® ArrayScan HCS System permit the subcellular location and concentration of fluorescently tagged moieties to be detected and quantified, both statically and kinetically. See also, U.S. Pat. No. 5,989,835, incorporated herein by reference in its entirety.

Markers other than fluorescent markers may be used, and markers need not be fused recombinantly to the aggregating protein.

For example, the protein can usefully be fused recombinantly to a tag that is recognized, and can thus be stained specifically by, an antibody.

Such tags include, for example, a myc tag peptide, the Xpress epitope, detectable by anti-Xpress antibody (Invitrogen Corp., Carlsbad, Calif., USA), the V5 epitope, detectable by anti-V5 antibody (Invitrogen Corp., Carlsbad, Calif., USA), FLAG® epitope, detectable by anti-FLAG® antibody (Stratagene, La Jolla, Calif., USA).

Other useful tags include, e.g., polyhistidine tags to facilitate purification of the recombinant fusion protein aggregant by immobilized metal affinity chromatography, for example using NINTA resin (Qiagen Inc., Valencia, Calif., USA) or TALON™ resin (cobalt immobilized affinity chromatography medium, Clontech Labs, Palo Alto, Calif., USA); a calmodulin-binding peptide tag, permitting purification by calmodulin affinity resin (Stratagene, La Jolla, Calif., USA), and glutathione-S-transferase, the affinity and specificity of binding to glutathione permitting purification using glutathione affinity resins, such as Glutathione-Superflow Resin (Clontech Laboratories, Palo Alto, Calif., USA), with subsequent elution with free glutathione.

Without intending to be bound by theory, it is possible that HD-nonspecific oligonucleotides that have an inhibitory effect on protein aggregation may be found to associate physically with the misassembled proteins. Isolating the protein aggregant under conditions suitable for continued binding of the oligonucleotide may thus permit enrichment for those oligonucleotides that have greatest affinity for the protein aggregant. See Kazantsev et al., Nature Genetics 30:367-76 (2002), incorporated herein by reference in its entirety.

Markers need not be fused recombinantly to the protein aggregant. For example, the protein aggregant can be marked by subsequent staining.

In other embodiments of the method of this aspect of the present invention, the oligonucleotide may be labeled.

Labeling the oligonucleotide is particularly useful for purposes of measuring, and normalizing to, the amount of oligonucleotide that enters the cells being assayed. Labeling of the oligonucleotides also permits the intracellular and extracellular distributions of the oligonucleotides to be assayed.

Typically, when the oligonucleotide is labeled, the protein aggregant is also labeled, since the subcellular distribution of oligonucleotide and protein aggregant may differ and provide complementary information.

The oligonucleotides may, for example, be labeled with a radionuclide, a fluorophore, or a visualizable hapten. When labeled with a radionuclide, the oligonucleotide's subcellular localization may be detected, e.g., using xray film or a phosphorimager. When labeled with a fluorophore, the oligonucleotide is typically labeled with a fluorophore having excitation and/or emission spectrum distinguishable from that optionally used to label the protein aggregant, and the oligonucleotide position and concentration is monitored using appropriate fluorescence detection devices.

The oligonucleotides may be labeled during or after synthesis. As described above, the label can be localized to the 5′ and/or 3′ terminus. In addition or in the alternative, the label can be positioned within the oligonucleotide.

When assayed in vitro, the cells used in the methods of this aspect of the invention are typically clonal lines that identically express the protein aggregant. The protein aggregant can be expressed from the cell's chromosome, either from its native locus or from another location into which an engineered construct has been integrated, or from an episomal construct.

When the cells are assayed in culture, the oligonucleotides to be tested for their ability to disrupt protein aggregation can be introduced into the cells by well-known transfection techniques.

Given the short length of the oligonucleotides, the oligonucleotides can be introduced passively, likely by endocytotic mechanisms, without further facilitation.

Alternatively, chemical transfection means can be employed.

For chemical transfection, DNA can be coprecipitated with calcium phosphate or introduced using liposomal and nonliposomal lipid-based agents. Commercial kits are available for calcium phosphate transfection (CalPhos™ Mammalian Transfection Kit, Clontech Laboratories, Palo Alto, Calif., USA), and lipid-mediated transfection can be practiced using commercial reagents, such as LIPOFECTAMINE™ 2000, LIPOFECTAMINE™ Reagent, CELLFECTIN® Reagent, and LIPOFECTIN® Reagent (Invitrogen, Carlsbad, Calif., USA), DOTAP Liposomal Transfection Reagent, FuGENE 6, X-tremeGENE Q2, DOSPER, (Roche Molecular Biochemicals, Indianapolis, Ind. USA), Effectene™, PolyFect®, Superfect® (Qiagen, Inc., Valencia, Calif., USA). Other types of polycations, cationic lipids, liposomes, and polyethylenimine (PEI) are known and may be used.

Mechanical means may also be used, such as electroporation, biolistics, and microinjection. Protocols for electroporating mammalian cells can be found online in Electroprotocols (Bio-Rad, Richmond, Calif., USA) (http://www.bio-rad.com/LifeScience/pdf/New_Gene_Pulser.pdf). For particle bombardment, see e.g. Cheng et al., Proc. Natl. Acad. Sci. USA 90(10):4455-9 (1993); Yang et al., Proc. Natl. Acad. Sci. USA 87(24):9568-72 (1990).

See also, Norton et al. (eds.), Gene Transfer Methods: Introducing DNA into Living Cells and Organisms, BioTechniques Books, Eaton Publishing Co. (2000) (ISBN 1-881299-34-1), incorporated herein by reference in its entirety.

Each oligonucleotide of distinct sequence and/or composition may be assayed individually, and its effectiveness in disrupting or preventing protein aggregation compared to that of other oligonucleotides. In addition or in the alternative, pools of oligonucleotides may be tested, either to facilitate initial screening or to identify combinations of oligonucleotides with additive or synergistic effect in disrupting huntingtin aggregations.

In the methods of this aspect of the invention, the oligonucleotides typically will be included within compositions suitable for introduction into cell culture, such as buffered aqueous compositions. Depending upon the duration of the assay, which typically ranges from hours to days, the oligonucleotides may preferably be formulated as sterile aqueous compositions.

Typically, but not invariably, the cells to be tested will be tested in a serum-free medium to prevent adventitious sequestration of the oligonucleotide by proteins in the medium.

After introduction of the oligonucleotide into the cells, the degree of protein aggregation is assessed and the efficacy of the oligonucleotide in disrupting or preventing protein aggregation determined. The efficacy may be measured statically, at any of a variety of time points, or kinetically, and various metrics of efficacy may be used.

For example, the degree of aggregation may measured as the total volume of protein aggregation within the cell at a particular time point after administration; as the number of separately distinguishable aggregates, such as “pinpoint aggregates”; as the greatest density of protein aggregation within the cell at a particular time point after administration; as the difference between greatest and least density of protein aggregation within the cell at a particular time point after administration. For kinetic assays, the effective degree of disruption may be measured as the rate at which the density, or volume, of aggregation dissipates in one or more regions of the cell. The choice among such metrics will be dictated, in part, by the cell type and aggregants selected for assay, and is well within the skill in the art.

The assay method may, and typically will, be repeated, until one or more oligonucleotides, alone or in combination, are identified that possess the desired degree of efficacy.

Other in vitro assays may also be used in this aspect of the invention.

Under some circumstances, protein aggregation can lead to cell death, and oligonucleotides able to inhibit or disrupt aggregation can be identified by their ability to inhibit cell death. See, e.g., Carmichael et al., Proc. Nat'l Acad. Sci. USA, 97:9701-9705 (2000).

Although oligonucleotides effective in disrupting or preventing aggregation will typically be chosen through in vitro assays such as those set forth above and in the Examples below, in other embodiments of this aspect of the invention the oligonucleotides will be assayed in vivo using an animal model of protein aggregation. In such in vivo assays, the efficacy of the oligonucleotide can be assessed by using clinical indicia of efficacy, such as diminution or delay of symptoms. In non-human animals, efficacy can also be assessed using post-mortem assays following sacrifice. A variety of such assays are described in the Examples that follow. See also Kazantsev et al., Nature Genetics 30:367-76 (2002), incorporated herein by reference in its entirety.

In a further aspect, the invention provides methods of treating human and animal subjects having Huntington's disease. The method comprises administering an effective amount of a composition comprising at least one HD-nonspecific oligonucleotide species that disrupts or prevents aggregation of huntingtin, optionally in admixture with a pharmaceutically acceptable carrier or excipient.

The administered composition will comprise at least one oligonucleotide prior-demonstrated, either in vitro or in an in vivo model, to disrupt or prevent aggregation of huntingtin, and may include any of the structural modifications described above.

The composition will comprise at least one species of oligonucleotide, and may comprise at least 2, 3, 4, 5, 10, 20, 25, 30, 40 and even as many as 50 to 60 different species, which may differ from one another in any one or more of sequence, length, or composition (such as presence, location, and number of altered internucleobase bonds).

Pharmaceutically acceptable carriers and/or excipients are optionally, but typically, included and are chosen for suitability with the desired method of administration.

Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X), the disclosures of which are incorporated herein by reference in their entireties, and thus need not be described in detail herein.

Pharmaceutical formulations designed specifically for administration of nucleic acids are also well known.

For example, one exemplary carrier for use with the oligonucleotides of the present invention includes nucleic acids, or analogues thereof, that do not themselves possess biological activity per se but that are recognized by in vivo processes that would otherwise reduce the bioavailability of the active oligonucleotides, for example by degrading the active oligonucleotides or promoting their removal from circulation. The coadministration of the active oligonucleotide and carrier nucleic acids, typically with an excess of the inactive material, can result in a substantial reduction of the amount of nucleic acid recovered in the liver, kidney or other extracirculatory reservoirs, presumably due to competition between the inactive carrier and the nucleic acid for a common receptor. See Miyao et al., Antisense Res. Dev., 5:115-121 (1995); Takakura et al., Antisense & Nucl. Acid Drug Dev., 6:177-183 (1996).

The pharmaceutically acceptable carrier and/or excipient may be liquid or solid and is chosen based, at least in part, upon the desired route of administration so as to provide for the desired bulk, consistency, etc., when combined with the oligonucleotides and the other components of a given pharmaceutical composition.

Routes of administration useful in the practice of this aspect of the invention include both enteral and parenteral routes, including oral, intravenous, intramuscular, subcutaneous, inhalation, topical, sublingual, rectal, intra-arterial, intramedullary, intrathecal, intraventricular, transmucosal, transdermal, intranasal, intraperitoneal, intrapulmonary, and intrauterine.

In treating Huntington's disease, certain routes of administration will require passage of the oligonucleotide active across the blood-brain barrier.

A useful embodiment makes use of neutral liposomes that carry the oligonucleotides and that are decorated on the surface with several thousand strands of polyethyleneglycol (PEG) as described in Pardridge, U.S. Pat. No. 6,372,250. The surface coating prevents the absorption of blood proteins to the surface of the liposome and slows the removal of the liposomes from the blood. It also provides sites for the attachment of ligands recognized by the carrier-mediated transport and receptor-mediated transcytosis systems to allow passage of the liposomes across the blood-brain barrier. In some cases, the ligands mediate the uptake of the pegylated liposomes by cells through the receptor-mediated endocytosis system.

In another useful approach, the oligonucleotides of the present invention are conjugated to targeting moieties that effect the delivery of the oligonucleotides into nerve cells and their retrograde transport to the nerve cell bodies.

As further described in international patent publications WO 02/47730 and WO 00/37103, incorporated herein by reference in their entireties, the targeting moieties are neurotrophins—such as NGF, BDNF, NT-3, NT-4, NT-6, and fragments thereof—that effect the targeted internalization of the compound by nerve cells of various classes.

Other methods for treating affected neuronal cells located in the brain utilize an implantable device such as an indwelling catheter through which the oligonucleotides, in an appropriate formulation, can be infused directly onto the neuronal cells. Alternatively, the oligonucleotide formulation is administered intranasally, e.g., by applying a solution containing the oligonucleotides to the nasal mucosa of a patient. This method of administration can be used to facilitate retrograde transport of the oligonucleotides into the brain. The oligonucleotides can thus be delivered to brain cells without subjecting the patient to surgery. See U.S. Pat. Nos. 5,624,898 and 6,180,603, the disclosures of which are incorporated herein by reference in their entireties.

In another, less preferred, alternative method, the oligonucleotides are delivered to the brain by osmotic shock according to conventional methods for inducing osmotic shock.

Other delivery systems and carriers can be selected that maximize delivery to neuronal cells in the central nervous system, especially in the brain. Such delivery systems and carriers are known to those of skill in the art. These delivery systems include liposomes, foams, wafers, gels and fibrin clots and the like. Delivery systems also include implantable devices such as indwelling catheters and infusion pumps. The delivery method can be selected depending on the location and type of neuronal cells to be treated.

The oligonucleotides of the invention are administered and dosed in accordance with standard medical practice, taking into account the clinical condition of the individual patient, the site and method of administration, scheduling of administration, patient age, sex, body weight and other factors known to medical practitioners.

The pharmaceutically “effective amount” for purposes herein is thus determined by such considerations as are known in the art. The amount is effective either to achieve improvement in clinical signs and/or symptoms—including but not limited to decreased levels of misassembled or aggregated huntingtin, or improvement or elimination of symptoms and other clinical endpoints—or to delay onset of or progression of signs or symptoms of disease, as are selected as appropriate clinical indicia by those skilled in the art. Cure is not required, nor is it required that improvement or delay, as above described, be achievable in a single dose.

The pharmaceutical composition is preferably administered in an amount effective to reverse protein misassembly and aggregation by at least about 10%, 20%, 30%, 40%, even at least about 50%, 60%, 70%, most preferably at least about 80-100%, although such dramatic effect is not required. It is preferred that the amount administered is an amount effective to maximize reversal of huntingtin protein misassembly and aggregation while minimizing toxicity.

The dosage can vary depending on the number of cells affected, the location of the cells, the route of administration, the delivery mode, whether treatment is localized or systemic, and whether the treatment is being used in conjunction with other treatment methodologies. Dosages can be determined using standard methodologies. Those skilled in the art can determine appropriate dosages and schedules of administration depending on the situation of the patient.

The composition is preferably administered until reversal of huntingtin protein misassembly and aggregation is obtained. Preferably the composition is administered from about 2 days up to a year, although chronic lifetime administration is not precluded. Advantageously, the time of administration can be coupled with other treatment methodologies. The oligonucleotide treatment may be applied before, after, or in combination with other treatments such as surgery or treatment with other agents. The length of time of administration can be varied depending on the treatment combination selected.

All references cited herein are hereby incorporated by reference.

The following are examples that illustrate the methods and compositions of this invention. These examples should not be construed as limiting: the examples are included for the purposes of illustration only.

### EXAMPLE 1

### Administration of Chimeric RNA/DNA Oligonucleotides Comprising a DNA Sequence Having at Least One Mismatch with Respect to the HD Gene Alters the HD Genetic Sequence

Two lymphoblastoid cell lines, obtained from Dr. Lance Whaley (Emory University), harbor a HD gene exon 1 with CAG/CAA (n=84) expansion tract and a CAG (n=24) length, respectively.

Using the procedure of Cole-Strauss et al. Nucleic Acids Research 27(5): 1323-1330 (1999), the disclosure of which is hereby incorporated by reference, cell-free extracts are prepared from these two cell lines. The extracts do not contain significant nuclease activity, which can skew the repair results by destroying either the target plasmid or the chimera. The extract is mixed with a plasmid containing a point mutation in the gene conferring kanamycin resistance at position 4021. See Cole-Strauss et al. Nucleic Acids Research 27(5): 1323-1330 (1999).

Two new chimera designs that enable higher frequencies of conversion in cell-free extracts (Gamper et al., Biochemistry 39: pp. 5808-5816 (2000), the disclosure of which is hereby incorporated by reference) and in cultured cells have been designed and tested. Both contain a mismatched base pair, which upon target hybridization forms a single mispairing (on the all-DNA strand). A second modification centers around the contiguous stretch of RNA residues on one strand. Each chimera is tested with the HD extracts from lymphoblastoid cell lines in the system designed to correct a point mutation in the kan⁵ gene. As shown in Table I, kan^{r} colonies are observed using either chimeric structure. Design II is clearly more effective in catalyzing the repair of the mutation. In addition, both extracts contain similar levels of repair activity. Ampicillin resistant colonies are used to normalize electroporation efficiencies and Table I represents an average of 5 independent experiments.

A similar strategy is used to measure the capacity of the B cell extract to catalyze the insertion of a T residue in a plasmid containing a frameshift mutation at position 208. See Cole-Strauss et al. Nucleic Acids Research 27(5): 1323-1330 (1999). Correction of this mutation confers tetracycline resistance to bacterial colonies bearing the wild-type plasmid. As shown in Table II, the frameshift mutation is also repaired by either chimera, but the difference between the two constructs is less dramatic. Based on these results, it appears that lymphoblastoid cells containing expanded and normal stretches of CAG repeats contain the necessary enzymatic activities to promote gene repair (or mutagenesis) of point and frameshift base targets.

Cell extracts of all cell lines described herein or that can be used for work relating to HD are checked for nuclease activity and for repair activity (or mutagenesis) by a method described above.

Transfection conditions of these lymphoblastoid cells are found to be optimal when the liposomal formulation, Lipofectamine, was used. Over 60% of the lymphoblastoid cells (CAG n=20) become labeled fluorescently by the uptake of an FITC-conjugated chimera.

The objective herein is to alter a CAG triplet of HD gene exon 1 to CTG, a change that would convert a glutamine residue to leucine. As shown in Table IIIb set forth at the end of this Example and in FIG. 3, the chimera design relies on the optimal structure determined from the cell-free extract experiments and is named HD1, which can be one of two chimeras (Table IIIb).

The lower case letters represent RNA residues and an internal A/A mismatch is designed within the sequence of the chimera. The isolated genomic DNA is amplified by PCR and the resulting fragment analyzed by Allele Specific PCR (ASPCR). The strategy is to employ the same leftward primer used to generate the PCR product. The rightward primer is ASPCR specific-designed so that the unchanged site will not provide a template for the rightward primer: two mismatches exist at the end. In contrast, a corrected sequence will have only a single unpaired base at the penultimate location (FIG. 4). ASPCR experiments can be done to show that a chimera HD1 can catalyze the conversion of CAG→CTG triplets at certain sites within the repeat regions. Also, the PCR products can be treated with PvuII to determine if an RFLP has been created.

A second cell line, obtained from Dr. Leslie Thompson (UCLA), is also employed. This cell line is a PC12 cell line which contains a HD gene exon 1 with a poly Q tract of 20. The same ASPCR analysis described above is used to detect CAG→CTG conversion events. PC12 cells (3×10⁵ cells/well-6 well plate) are treated with two concentrations of a HD1 chimeric RNA/DNA oligonucleotide (1 μg and 2 μg). The chimeric RNA/DNA oligonucleotides are transfected by lipofection (Lipofectamine 1 μg) and incubated for 6 hours. The cells are washed with PBS and fresh medium is added and cells are incubated for an additional 18 hours. Genomic DNA is isolated and the target region amplified by PCR. These fragments provide the template for ASPCR. ASPCR experiments show that CAG→CTG conversion can occur.

Another cell line, obtained from Dr. Laising Yen (Harvard University) is used. These are 293 cells with integrated copies of the HD gene exon 1 containing 84 CAG repeats. The same transfection protocol is used as described above, except that another set of chimeras (named HDII, which can be one of two chimeras) is employed (Table IIIb). The objective of these experiments is to interrupt the CAG repeats by insertion of an A residue, thereby causing a frameshift in the chromosomal HD gene.

After genomic DNA isolation, a 350 base pair PCR fragment is generated from the whole population and cloned via TA- tailing into a plasmid for direct DNA sequence analyses. The control and the experimentally treated cells produce sequence data showing insertions. In conjunction with the results reported above, it has been determined that the frequency of inducing frameshift mutation is approximately 10-20 fold lower than the frequency of nucleotide exchange.

In another experiment with these cells, a chimera HD1 is used to target 293 cells (n=84) to induce a CAG→CTG point mutation. Using the same approach outlined above for B cells, ASPCR results (FIG. 5) indicate the presence of sequence alterations. Note that the control lane, but not the H₂O lane, has a faint band that may represent mutagenesis of the integrated plasmid or artefactual background. In any case, the level is substantially lower than samples exposed to a HD1 chimera. The data are presented in groups of three as time is devoted to optimizing transfection conditions (see chart below FIG. 5). We find that the best results are observed when Lipofectamine is used as a carrier. Genomic amplification of samples judged positive by ASPCR are tested for RFLP by digestion with PvuII.

In summary, B cells containing an N=20 CAG repeat are altered in the HD gene by chimera-directed nucleotide exchange. A CAG→CTG point mutation alteration is confirmed by ASPCR and RFLP analyses. A 293 cell line containing an N=84 CAG repeat is also targeted for both frameshift mutation and point mutation. 293 (N=84 CAG repeat number) cells targeted for CAG→CTG alteration produces a strong signal that conversion has occurred, according to ASPCR analysis.

Primers are designed that encompass both unique regions 5′ and 3′ relative to the CAG repeat for PCR amplification. Detection of converted bases is carried out by ASPCR as described. In addition, cells are separated, after the transfection and initial growth periods, into 96-well plates and are grown to allow expansion for several days. Each group of cells provides the source of genomic DNA for PCR amplification. DNA sequence analyses are conducted directly on these PCR amplified fragments such that converted cells are more evident during the expansion process in some of the wells. Fluorescently tagged antibodies directed against Huntingtin are used to measure changes in aggregation levels and/or cellular localization.

A new targeting oligonucleotide has evolved from structure function relationship studies (see Gamper et al. Biochemistry 39:5808-5816 (2000)). Extensions of the RNA residues are placed near the 5′ and 3′ ends of a single stranded structure forming a modified double-hairpin known as “the cradle.” In biochemical (cell-free extract) assays using cells from various sources (including HD B cells), a four-fold increase in correction activity is observed.

Another set of cell lines is provided by Dr. George Lawless. CHO cells with integrated copies of HD gene exon 1 with approximately 103Q repeats fused to GFP as a fusion construct encoding HD gene exon 1 Q103-GFP produce a visible GFP aggregation at the nuclear membrane, detectable by microscopy, whereas CHO cells with integrated copies of fusion constructs encoding HD gene exon 1 Q24-GFP in CHO cells do not produce a visible GFP aggregation at the nuclear membrane. One set of chimeras are designed to convert CAG to AAG (lysine) or (CCG) (proline) thereby maintaining fusion gene expression but hopefully reducing the aggregation at the nuclear membrane and increasing the percentage of GFP found dispensed in the cytoplasm. A second set of chimeric oligonucleotides direct CAG→TAG nucleotide exchange. A 58 mer that targets a CAG to TAG change in HD exon 1 is shown in FIG. 6a. This chimera can work on any cell lines. Result of gene alteration using this chimera on a cell line bearing HD gene exon 1 is shown in FIG. 6b. The change is made at the particular CAG shown in FIG. 6b due to sliding of repeat region, a phenomenon that can occur with the methods of this invention.

This latter set of chimeras and cradle conformers should direct a nucleotide exchange such that the CAG repeat is altered to at least one stop codon. In such case, GFP will not be translated and a reduction in fluorescence will be observed. This difference is measurable by microscopy with a FITC cube filter.

Since the repeat sequence is not a single CAG but rather . . . CAACAACAGCAGCAACAG . . . , a more directed targeting approach can be taken. Based on the repeat length, there are 15 possible “best fit” target sites in the gene. Cloning cylinders can be placed in regions within the culture dish where fluorescence is reduced. Populations of cells recovered from these cylinders are enriched for converted cells, making the detection of DNA alteration by sequence analyses easier. Coupled with the visual aspects of GFP-aggregation, DNA sequence (without a background of unconverted nucleotides) provides evidence of gene alteration in animal models.

TABLE I

| | | | | Amp^{r} | Kan^{r} |
|---|---|---|---|---|---|
| | Plasmid | Chimera | Extract | Colonies | Colonies |
| 1 | + | I | 0 | 239 | 0 |
| 2 | + | II | 0 | 308 | 0 |
| 3 | + | − | 0 | 270 | 0 |
| 4 | − | I | 0 | 0 | 0 |
| 5 | − | II | 0 | 0 | 0 |
| 6 | − | − | 2.5 λ | 0 | 0 |
| 7 | + | − | 2.5 λ | 305 | 0 |
| 8 | − | I | 2.5 λ | 290 | 0 |
| 9 | + | I | 2.5 λ | 247 | 662 |
| 10 | + | II | 2.5 λ | 237 | 1089 |
| 11 | + | I | 2.5 λ | 278 | 673 |
| 12 | + | II | 2.5 λ | 283 | 1247 |

TABLE II

| | | | | Amp^{r} | Tet^{r} |
|---|---|---|---|---|---|
| | Plasmid | Chimera | Extract | Colonies | Colonies |
| 1 | + | I | 0 | 233 | 0 |
| 2 | + | — | 2.5 λ | 291 | 0 |
| 3 | + | I | 2.5 λ | 275 | 103 |
| 4 | + | II | 2.5 λ | 313 | 213 |
| 5 | + | I | 10 λ | 266 | 96 |
| 6 | + | II | 10 λ | 281 | 147 |

TABLE IIIA

| | |
|---|---|
| HDA3T/53 | [SEQ ID NO:1] |

This single-stranded (ss) oligonucleotide has a
mismatch relative the HD gene on the last base to
avoid acting as a primer in PCR.
5′ C*G*A*GTCCCTCAAGTCCTTCCAACAGCTGCAACAGCAACAACAGC
AGCAAC*A*G*A 3′

| | |
|---|---|
| Kan uD12T/25G | [SEQ ID NO:2] |

This oligonucleotide has all thioate linkages
5′T*T*G*T*G*C*C*C*A*G*T*C*G*T*A*G*C*C*G*A*A*T*A*G*C 3′

| | |
|---|---|
| Kan uD3T/25G | [SEQ ID NO:3] |

This oligonucleotide has 3 thioates on each end
5′T*T*G*TGCCCAGTCGTAGCCGAAT*A*G*C 3′

| | |
|---|---|
| Kan uRD3/25G - (3) 2′O-Me links | [SEQ ID NO:4] |

on each end
This oligonucleotide has three 2′O-Me
modifications on each end
5′uugTGCCCAGTCGTAGCCGAATagc 3′

| | |
|---|---|
| Kan uR/25G | [SEQ ID NO:5] |

This oligonucleotide has all RNA 2′O-Methyl
modifications
5′uugugcccagucguagccgaauagc 3′

| | |
|---|---|
| Kan uR/15G | [SEQ ID NO:6] |

This oligonucleotide has all RNA 2′O-Methyl
modifications
5′gcccagtcgtagccg 3′

| | |
|---|---|
| Kan uD7T/15G | [SEQ ID NO:7] |

This 15-mer has all thioate linkages
5′G*C*C*C*A*G*T*C*G*T*A*G*C*C*G 3′
LEGEND:
* denotes phosphorothioate linkages
lower case - 2′O Methyl RNA nucleobase
upper case - DNA nucleobase

TABLE IIIB
HD1 Chimeras

| | |
|---|---|
| uGTCGTCGTCGTCGACGTCGTCGTCGTCu | [SEQ ID NO:8] |

u u
u u
ucagcag3′ 5′cag cag cag cag cag cag cag u

| | |
|---|---|
| 5′ CTG-CTG-CTG-CTG-CAG-CTG-CTG- | [SEQ ID NO:9] |

CTG-CTG uuuu-cag-cag-cag-cag-cag-
cag-cag-cag-cag-CAG-CAG-uuuu-CTG-
CTG 3′

HDII Chimeras
(Cause a frameshift mutation in the chromosomal RD
gene exon 1 by insertion of a basepair.)

| | |
|---|---|
| uGTCGTCGTCGTCTGTCGTCGTCu | [SEQ ID NO:10] |

u u
u u
ucag3′ 5′cagcagcag u cag cag cag u

| | |
|---|---|
| 5′ CTG-CTG-CTG-CTG-CTAG-CTG-CTG- | [SEQ ID NO:11] |

CTG-CTG uuuu-cag-cag-cag-cag-cag-
cag-cag-cag-cag-CAG-CAG-uuuu-CTG-
CTG 3′

LEGEND:
DNA residues are in upper case;
RNA residues are in lower case.

### EXAMPLE 2

### Administration of Modified Single Stranded Oligonucleotides Comprising a DNA Sequence Having at least One Mismatch with Respect to the HD Gene Decreases Aggregate Formation of HD Protein in Cell Culture

PC12 neuronal cell lines, provided by L. Thompson (UCI), are used. See Boado et al. J. Pharmacol. and Experimental Therapeutics 295(1): 239-243 (2000), the disclosure of which is hereby incorporated by reference. This PC12 cell line has a construct (see Kazantsev et al. Proc. Natl. Acad. Sci. USA 96: 11404-09 (1999), the disclosure of which is hereby incorporated by reference) integrated into its genome. These cells thus contain an engineered HD gene exon 1 containing alternating, repeating codons . . . CAA CAG CAG CAA CAG CAA . . . fused to an enhanced GFP (green fluorescent protein) gene. Hence, expression of this gene leads to the appearance of green fluorescence co-localized to the site of protein aggregates. The HD gene exon 1-GFP fusion gene is under the control of an inducible promoter regulated by muristerone. A particular construct has approximately 46 glutamine repeats (encoded by either CAA or CAG) . Other constructs have, for example, 103 glutamine repeats.

These cells are transfected with the modified single stranded oligonucleotide HDA3T/53 (a 53 mer) (see Table IIIa), which can alter the HD gene sequence and in fact is designed to convert a CAG→CTG in the HD gene exon 1 that encodes the polyQ stretch. This modified single stranded oligonucleotide (HDA3T/53) is modified at each terminus bearing phosphorothioate linkages in the three terminal bases. HDA3T/53 is an oligonucleotide for targeted alteration of the genetic sequence of the Huntington's disease gene, which comprises a single-stranded oligonucleotide having a DNA domain, the DNA domain having at least one mismatch with respect to the genetic sequence of the Huntington's disease gene to be altered.

FIG. 7 shows an outline of this experiment. These PC12 cells are grown in DMEM, 5% Horse serum (heat inactivated), 2.5% FBS and 1% Pen-Strep, and maintained in low amounts on Zeocin and G418. 24 hours prior to transfection, the cells are plated in 24-well plates coated with poly-L-lysine coverslips, at a density of 5×10⁵ cells/ml in media without any selection. Transfection conditions are optimized using lipofectAMINE 2000 (“LF2000”) at varying ratios of LF2000 to oligonucleotide. Cells are also treated with various non-specific oligonucleotides as a control (see Example 3). LF2000 is incubated with Opti-Mem I (serum-free medium) for 5 minutes. The oligonucleotide is added and further incubated for 20 minutes at room temperature. The lipid/DNA mixture is applied to the cells and incubated at 37° C. overnight. Muristerone is added after the overnight incubation to induce the expression of HD gene exon 1-GFP.

The data are acquired on a Zeiss inverted 100M Axioskop equipped with a Zeiss 510 LSM confocal microscope and a Coherent Krypton Argon laser and a Helium Neon laser. Samples are loaded into Lab-Tek II chambered coverglass system for improved imaging. The number of Huntingtin-GFP aggregations within the field of view of the objective is counted in 7 independent experiments.

Results and Conclusions

Fields of view from seven independent transfections of PC12 cells harboring an HD gene exon 1-GFP in which the exon 1 encodes approximately 103 glutamine residues are analyzed. Representative pictures from these experiments are displayed in FIG. 8 (A-D). FIG. 8A displays a typical field of view from untransfected PC12 cells while FIG. 8B-D illustrate fields of view from cells treated with HDA3T/53.

To gain an approximation of the number of “pinpoint aggregates” present, several scientists are requested to perform an unbiased count of Huntingtin-GFP fusion protein aggregates in various fields from control and treated cell populations.

The results, shown in FIG. 9, show that a 60% reduction in these specific aggregate types occur repeatedly. The decrease in Huntingtin-GFP fusion protein aggregate number appears to be maximized at 1 μg of modified single stranded oligonucleotide (i.e., HDA3T/53) added as an increase in concentration does not lead to improved results. Molecular analyses of these cells are performed to show a correlation between aggregate reduction and changes at the DNA level.

The same experiment is repeated in PC12 cells containing Q46/GFP (i.e., HD gene exon 1 GFP fusion gene in which there are approximately 46 glutamine repeats in HD gene exon 1).

Other experiments are performed with a range of concentrations of either modified single stranded oligonucleotides or chimeric RNA/DNA oligonucleotides to measure the effect of oligonucleotide concentration on the extent of Huntingtin protein-GFP fusion aggregation. These data also indicate the optimal dose of oligonucleotide to maximally reduce aggregation.

In a further experiment, a short (10-15 base), single stranded “trapper” oligonucleotide completely complementary to the second strand of the helix (non-targeted strand) is also used. The addition of a trapper oligonucleotide increases the frequency of conversion using a modified single stranded oligonucleotide at least 10-fold. See FIG. 10. This short oligonucleotide consists of modified nucleic acid residues, for example PNA (peptide nucleic acid) or LNA (Locked Nucleic Acid), which elevate stability and extend the half-life of the repair complex or a double D-loop structure. This trapper oligonucleotide can be used in conjunction with a molecule such as HDA3T/53. Alternatively, modified single stranded oligonucleotides complementary to a molecule such as HDA3T/53 are used with a trapper oligonucleotide that hybridizes to the opposite strand of the duplex.

The “clones” or cells that survive after treatment with an oligonucleotide are analyzed by DNA sequencing to determine whether there are specific, targeted alterations in the Huntington protein-GFP fusion gene.

A cell line, PC12/pBWN:httexl(Q103), containing the first exon of Huntingtin including the Q103 repeat, fused to the eGFP (enhanced GFP) gene (gift of Dr. Erik Schweitzer, UCLA). The promoter directing expression of the Huntingtin eGFP fusion is regulated by ecdysone analogs. These cells are useful because after induction, aggregate formation is overwhelming and other cellular activity is observed; eventually, the cells die. Hence, a disruption in aggregate formation, through a specific sequence alteration or nonspecific effect will ultimately prolong cell life and proliferation with sustained green fluorescence. Careful measurements of extending cell life are made.

Modified single stranded oligonucleotides, as well as chimeric RNA/DNA oligonucleotides, designed to convert a CAG triplet of HD exon 1 to CTG are tested for the ability to reduce aggregate formation. The effect of non-specific oligonucleotides (an oligonucleotide that is not specific for the HD gene) is also tested. The toxicity of all the oligonucleotides is also tested using viability staining. A short (10-15 base), single stranded “trapper” oligonucleotide completely complementary to the second strand of the helix (non-targeted strand) is also used in the PC12/pBWN:httexl(Q103) assay system.

The “clones” or cells that survive after treatment with an oligonucleotide are analyzed by DNA sequencing to determine whether there are specific, for example oligonucleotide-directed, alterations in the Huntingtin protein-GFP fusion gene.

Also, cells from HD patients are analyzed directly for gene conversion events. Molecular analyses are carried out by allele specific-PCR or ASPCR, a sensitive detection system developed for chimera-directed gene repair in our laboratory. Sensitivity levels approaching 0.1% to 0.5% signaling successful genomic targeting are attainable.

### EXAMPLE 3

### Administration of a Non-Specific Oligonucleotide, which does not Hybridize to the HD Gene, Decreases Aggregate Formation of HD Protein in Cell Culture Studies

As part of the experiments of Example 2, an excess of single stranded DNA molecules having no sequence complementarity to the target HD gene are added to PC12 cells bearing an HD gene exon 1-GFP fusion gene; these are non-specific oligonucleotides (oligonucleotides that do not hybridize to DNA encoding Huntingtin protein or its complement), modified in a similar fashion as the modified single stranded oligonucleotide of Example 2 at each termini.

The PC12 cells (Boado et al. J. Pharmcol Exp Ther. 295(1): 239-243 (2000)) contain a CAG or CAA repeat of approximately 46 or 103 in the CAG/CAA tract, encoding the poly Q tract, in the first exon of the HD gene fused to an eGFP (enhanced GFP) fusion reporter construct. See Example 2 and Kazantsev et al. Proc. Natl. Acad. Sci. USA 96: 11404-09 (1999). When these cells are treated (transfected) with oligonucleotides that are not specific for the HD gene prior to the induction of fusion gene expression, the number of Huntingtin-GFP fusion protein aggregates formed during the course of the next 72 hours is significantly reduced (FIGS. 11-12). As described in Example 2, the HD gene exon 1-GFP fusion gene in these PC12 cells is under the control of an inducible promoter regulated by muristerone.

The protocol described in Example 2 for these PC12 cells (Boado et al. J. Pharmcol Exp Ther. 295(1): 239-243 (2000)) is essentially followed in this Example. See also FIG. 7.

The experiment can also be done in a different way. The non-specific oligonucleotides can be added to the PC12 cells 48 hours after the induction of gene expression by addition of muristerone; and 48-72 hours later, the cells are visualized by confocal microscopy.

In the absence of oligonucleotide, activation of the promoter leads to high levels of Huntingtin-GFP fusion gene expression and, subsequently, the appearance of Huntingtin-GFP fusion protein aggregates (bright pinpoints), visible in FIG. 11A and FIG. 12A.

A visible reduction in the presence of Huntingtin-GFP fusion protein aggregates is observed in the presence of an oligonucleotide that does not hybridize to the HD gene (“non-specific” or “HD non-specific”) (Kan uD3T/25G; see Table IIIa for structure and sequence). See FIG. 11B and Table IIIa. Kan uD3T/25G is a 25 mer single stranded DNA oligonucleotide with 3 phosphorothioates on each terminus. FIG. 11C shows that administration of a 25 mer HD non-specific single stranded oligonucleotide with all phosphorothioate linkages (Kan uD12T/25G; see Table IIIa for structure and sequence) results in a reduction in Huntingtin-GFP fusion protein aggregates (same results are obtained with kan uD7T/15G, a 15 mer single stranded HD non-specific oligonucleotide with all phosphorothioate linkages). Note that the degree of reduction is actually similar for both oligonucleotides. FIGS. 11B and 11C are not shot at the same magnification. Reduction of Huntingtin-GFP fusion protein aggregate formation is also observed for Kan uRD3/25G (Table IIIa). See FIG. 12. However, two other non-specific oligonucleotides (kan uR/25G and kan uR/15G (Table IIIa)) have little to no effect. See FIG. 12. The reduction of aggregate formation due to the presence of Kan uRD3/25G is not as great as those observed due to the presence of Kan uD3T/25G or Kan uD12T/25G. The oligonucleotide Kan uRD3/25G is a 25 mer HD non-specific single stranded DNA oligonucleotide with three 2′-O-methyl RNA on each terminus. The oligonucleotide Kan uR/25G is a 25 mer HD non-specific single stranded oligonucleotide with all 2′-O-methyl RNA. The oligonucleotide Kan uR/15G is a 15 mer HD non-specific single stranded oligonucleotide with all 2′-O-methyl RNA.

In certain experiments, the above-described reduction in Huntingtin-GFP fusion protein aggregate formation effect is observed only when an excess (>25 μg) of an oligonucleotide that is not specific for the HD gene is transfected. The right amount of HD non-specific oligonucleotide required to reduce Huntingtin-GFP fusion protein aggregate formation may vary and can be easily determined.

This disaggregation effect is poorly observed when a chimeric RNA/DNA oligonucleotide that does not hybridize to the HD gene is used.

In summary, administration of a single stranded DNA that is specific for HD, such as HDA3T/53, which has three phosphorothioates at each terminus, results in significant reduction in formation of HD protein aggregates. Non-specific single stranded DNA, such as Kan uD3T/25G, which has three phosphorothioates at each terminus, or such as Kan uD12T/25G or Kan uD7T/15G, which are substituted with all phosphorothioates, are also effective (though perhaps less so than HDA3T/53) in reducing the formation of the number of HD protein aggregates. A single stranded DNA with 3 2′-O-methyl RNA at each terminus, such as Kan uRD3/25G, is less effective in reducing the number of HD protein aggregates than Kan uD3T/25G or Kan uD12T/25G. Non-specific double stranded chimeric RNA/DNA oligonucleotides are also less effective in reducing the number of aggregates. A single stranded oligonucleotide with all 2′-O-methyl RNA residues, such as Kan uR/25G or Kan uR15/G, has little to no effect.

Using this same experimental system, an oligonucleotide comprising different lengths, different base composition, or different base modification but which are not specific for the HD gene are examined to determine optimal length and composition for the disaggregation effect. Similarly, varying concentrations of such oligonucleotides and those described above are tested for aggregate reduction using the assay described herein. In this way, the optimal concentration of oligonucleotides of defined length and defined composition is determined.

### EXAMPLE 4

### Administration of Modified Single Stranded Oligonucleotides Comprising a DNA Sequence with at least One Mismatch with Respect to the HD Gene Alters the HD Genetic Sequence

Modified single stranded oligonucleotides (25^{mer} and 52^{mer}, each with three phosphorothioates at each terminus) shown in FIG. 13, as well as HDA3T/53, cause a CAG to CTG gene alteration in cells comprising a HD gene, or portion thereof, which encodes Huntingtin protein (or a portion thereof) having varying lengths of glutamine. The cells comprise an HD gene exon 1-GFP fusion construct which encodes a Huntingtin-GFP protein with approximately 20, 46 or 103 glutamine in its polyQ tract.

PC12 cells bearing HD gene exon 1-GFP fusion gene are transfected with the oligonucleotides described in FIG. 14 by liposome transfection. Two days later, extracts are made. Molecular analyses, such as PCR and TA cloning, are done. After genomic DNA isolation, a PCR fragment is generated and cloned via TA- tailing into a plasmid for direct DNA sequence analyses. Results of DNA sequence analysis of exemplary experiments are shown in FIG. 14. A CAG to CTG gene alteration event is effected by this method.

### EXAMPLE 5

### Administration of a Short Oligonucleotide Decreases Aggregate Formation of HD Protein in Cell Culture Studies

Further to the experiments of Examples 2 and 3, several other single stranded DNA molecules are added to PC12 cells bearing an HD gene exon 1-GFP fusion gene (see Examples 2 and 3); these are both specific (oligonucleotides that hybridize to DNA encoding Huntingtin protein or its complement) (the specific oligonucleotide may alter HD gene sequence) and non-specific oligonucleotides. The specific oligonucleotides are a 15 mer (HDA3T15 mer) and a 9 mer (HDA3T9 mer), each of which is modified with three phosphorothioate linkages in each terminus. The non-specific oligonucleotide is a 15 mer oligonucleotide comprising LNA residues (klo17LNA).

The sequences of these oligonucleotides are as follows, where “*” denotes a phosphorothioate linkage and a “+” is prefixed before an LNA residue:

| | |
|---|---|
| 5′ C*T*G*TTGCAGCTG*T*T*G 3′ | [SEQ ID NO:12] |

(HDA3T15mer)

| | |
|---|---|
| 5′ T*T*G*CAG*C*T*G 3′ | [SEQ ID NO:13] |

(HDA3T9mer)

| | |
|---|---|
| 5′ +C+T+CA+GG+AG+T+C+AG+G+TG 3′ | [SEQ ID NO:14] |

(klo17LNA)

Additional oligonucleotides are also tested, including a 25 mer mismatched to the target (i.e., does not hybridize to the HD gene), having 3 LNA on either end (Kan klo1); a 15 mer mismatched to the target (i.e., does not hybridize to the HD gene, containing all LNA modified bases (Kan klo2); a 15 mer mismatched to the target (i.e., does not hybridize to the HD gene), having 4 LNA on either end (kan klo3); and a 9 mer, all LNA (kan klo4):

| | |
|---|---|
| 5′ +T+T+GTGCCCAGTCGTAGCCGAAT+A+G+C3′ | [SEQ ID NO:15] |

(kan klo1)

| | |
|---|---|
| 5′ +G+C+C+C+A+G+T+C+G+T+A+G+C+C+G 3′ | [SEQ ID NO:16] |

(Kan klo2)

| | |
|---|---|
| 5′ +G+C+C+CAGTCGTA+G+C+C+G 3+ | [SEQ ID NO:17] |

(kan klo3)

| | |
|---|---|
| 5′+C+A+G+T+C+G+T+A+G3′ | [SEQ ID NO:18] |

(kan klo4)

PC12 cells (Boado et al. J. Pharmacol. and Experimental Therapeutics 295(1): 239-243 (2000)) are used. These particular PC12 cells contain a CAG or CAA repeat of approximately 103 in the CAG/CAA tract, encoding the poly Q tract, in the first exon of the HD gene fused to an eGFP (enhanced GFP) fusion reporter construct. This PC12 cell line has a construct (see Examples 2-3 and Kazantsev et al. Proc. Natl. Acad. Sci. USA 96: 11404-09 (1999)) integrated into its genome. These cells thus contain an engineered HD gene exon 1 containing alternating, repeating codons . . . CAA CAG CAG CAA CAG CAA . . . fused to a GFP gene. As described in Example 2, the HD gene exon 1-GFP fusion gene in these PC12 cells is under the control of an inducible promoter regulated by muristerone.

The protocol described in Example 2 for these PC12 cells (Boado et al. J. Pharmcol Exp Ther. 295(1): 239-243 (2000)) is essentially followed. See also FIG. 7. When these cells are treated (transfected) with HDA3T15 mer and HDA3T9 mer, which are oligonucleotides that can hybridize to the HD gene, prior to the induction of Huntingtin-GFP fusion gene expression, the number of Huntingtin-GFP protein aggregates formed during the course of the next 72 hours is significantly reduced (FIG. 15). 5 μg of the oligonucleotide is added to the PC12 cells (the oligonucleotide is added by transfection; see protocol in FIG. 7 and Example 2, for these PC12 cells) and the cells are incubated for 24 hours. Gene expression is then induced in the cells by the addition of muristerone. See protocol in FIG. 7 and Example 2 (for these PC12 cells). After the cells are incubated for 48 hours, the cells are analyzed by confocal microscopy. See protocol in FIG. 7 and Example 2 (for these PC12 cells). In the absence of oligonucleotide, activation of the promoter leads to high levels of fusion gene expression and, subsequently, the appearance of Huntingtin-GFP protein aggregates (bright pinpoints) visible in the “untransfected” and “untransfected 2” panels of FIG. 15.

A visible reduction in the appearance of Huntingtin-GFP protein aggregates is observed in the presence of HDA3T15 mer (approximately 55% decrease), HDA3T9 mer (approximately 55% decrease) and KanuD3T/25G (approximately 50% decrease), but not in the presence of klo17LNA (none of the oligonucleotides comprising LNA residues, shown above in this Example, reduces Huntingtin-GFP protein aggregate formation). KanuD12T/25G has a toxic effect on these cells (i.e., causes more cell death). See FIG. 15.

Accordingly, addition of short single stranded DNA molecules that are 9 mer or 15 mer having sequence complementarity to the target HD gene and having three phosphorothioates at the terminus of each molecule is effective in causing significant reduction in the formation of HD protein aggregates. Oligonucleotides comprising LNA residues and that are non-specific to the HD gene (i.e., does not hybridize to the HD gene) have no effect in reducing the formation of HD protein aggregates.

Using this same experimental system, oligonucleotides comprising different lengths, different base composition, or different base modification but which are or are not specific for the HD gene are examined to determine optimal length and composition for the disaggregation effect. Similarly, varying concentrations of such oligonucleotides and those described above are tested for aggregate reduction using the assay described herein. In this way, the optimal concentration of oligonucleotides of defined length and defined composition is determined.

### EXAMPLE 6

### A Cell Survival Assay for Detecting Disaggregation of Huntingtin Aggregates and/or Correction of the HD Gene

A cell line, PC12/pBWN:httex, containing the first exon of Huntingtin including the 103 polyglutamine repeats (each Q is encoded by either CAA or CAG; essentially alternating CAACAG), fused to the eGFP (enhanced GFP) gene (gift of Dr. Erik Schweitzer, UCLA) is used. This cell line has incorporated a construct with essentially alternating CAACAG encoding for the PolyQ tract (see Schweitzer et al., J. Cell. Science 96: 375-381 (1990); the disclosure of which is incorporated by reference herein). The promoter directing expression of the Huntingtin-eGFP fusion is regulated by ecdysone analogs. The cells bearing this ecdysone-regulated vector die upon induction with tebufenozide. These cells are useful because after induction, Huntingtin aggregate formation is overwhelming and other cellular activity is observed; eventually, the cells die. Hence, a disruption in Huntingtin aggregate formation, through a specific sequence alteration or nonspecific effect, or specific sequence alteration without disaggregation, ultimately prolong cell life and proliferation as indicated by sustained green fluorescence. Careful measurements of extending cell life are made.

Treating these cells with single stranded DNA molecules, specific for the HD gene (and which may alter HD gene sequence), causes disaggregation of the Huntingtin aggregates and/or gene correction, as well as increasing survival of these cells.

1×10⁵ cells are passaged in poly-D-lysine coated T25 flasks 4-5 days prior to transfection, as the cells have a slow growth rate. The cells are transfected by using 10 μg Lipofectamine2000 with 5 μg single stranded oligonucleotide (HDA3T9 mer, HDA1T9 mer or HDAT9 mer, the sequences of which are shown below) mixed with 500 μl Optimem.

| | | |
|---|---|---|
| HDA3T9mer: | 5′ T*T*G*CAG*C*T*G 3′ | [SEQ ID NO:13] |
| HDA1T9mer: | 5′ T*TGCAGCT*G 3′ | [SEQ ID NO:19] |
| HDAT9mer: | 5′ T*TGCAGCTG 3′ | [SEQ ID NO:20] |

where “*” denotes a phosphorothioate linkage.

The cells are induced 24 hours after transfection by the addition of 0.1 μM tebufenozide (day 1). Confocal microscopy photos are taken on days 2, 3, 6 and 7 post-induction.

On day 7 post-induction, there are about 1% cells surviving in flasks treated with HDA3T9 mer and HDAT9 mer (FIG. 16, parts a and b). However, by day 6 post-induction, untreated cells (ut) and cells transfected with HDA1T9 mer do not survive. See also FIG. 17.

FIG. 17 shows a PC12 cell survival quantitation graph. Cells survive in flasks treated with HDAT9 mer 4, 6 and 7 days post induction, a time when untreated cells do not survive.

Accordingly, single stranded DNA molecules, non-specific for the HD gene, cause disaggregation of Huntingtin protein aggregates in these cells, which is manifested in these cells as cell survival.

This cell system can be used for studying disaggregation of Huntingtin protein aggregates or alteration of HD gene by any agent, such as the oligonucleotides of this invention and oligonucleotides that are not specific to the HD gene.

### EXAMPLE 7

### Disruption of Aggregates Using HD-Specific and Using HD-Nonspecific Oligonucleotides

Experimental Procedures

Cell Culture, Transfection, and DNA Analyses

Lymphoblastoid cells containing CAG (n=16, 20) polyglutamine repeats are maintained in Iscove's Modified Dulbecco's medium (Life Technologies) containing 15% fetal bovine serum, 5 ml 200 mM glutamine, and 2.5 ml 10 mg/ml gentamycin sulfate (Life Technologies, Inc). For nucleotide exchange experiments, 10⁵ cells are seeded in a 24-well plate 24 hours prior to transfection.

Oligonucleotides HD3S/52 and HD3S/25 are delivered by lipofection using DOTAP (Roche) diluted with 20 mM Hepes, pH7.4; the optimal amount of cationic liposomes is fixed at 10 μg/ml per μg DNA. The oligonucleotides are diluted into 20 mM Hepes, pH7.4, mixed, and complexed with liposome at 22° C. for 30 min. The complex is then applied to the cells, which are harvested 48 hours later by centrifugation at 3000 rpm for 5 minutes.

The pellets are washed twice with 1×PBS, minus Ca²⁺ and Mg²⁺ (Life Technologies Inc.), followed by resuspension in 50 μl K buffer (50 mM KCl, 10 mM Tris, pH8.0, 0.5% tween20) and 10 mg/ml Proteinase K. The pellet is then incubated at 56° C. for 45 mins, and heat-inactivated at 95° C. for 10 mins.

PCR amplification of genomic DNA is carried out using a GC-RICH PCR system (Roche). The reaction contains 100 ng genomic DNA extract, primer HD-5 (5′-gatggacggccgctcagg) [SEQ ID NO:21] (200 mM), primer HD-3 (5′-gaggcagcagcggctgtg) [SEQ ID NO:22] (200 mM), 500 μM dNTP mix, 5×GC-RICH PCR reaction buffer with DMSO, 2M GC-RICH resolution solution, and 2U GC-RICH PCR system enzyme mix. PCR conditions are set at 95° C. for 3 minutes, 30 cycles at 95° C. for 30 sec, 55° C. for 30 sec, 68° C. for 2 mins, followed by elongation at 68° C. for 7 min and storing at 4° C.

The PCR product (322 bp) is visualized on a 1.5% agarose gel, and the samples are then purified using Qiagen PCR purification kits. The PCR product is then cloned into PCR®2.1 using the protocol from Original TA cloning kit (Invitrogen). These clones are analyzed for gene conversion by RFLP analysis using PvuII; digests that produce a 280 bp band are submitted for DNA sequencing using an automated ABI 310 capillary sequencer.

Protein Aggregate Analyses

PC-12 cells (a gift from Dr. L. Thompson, UCI) (PC12-103QeGFP) are maintained in DMEM, 5% horse serum (heat inactivated), 2.5% FBS, 1% Pen-Strep, 0.2 mg/ml zeocin, and 100 μg/ml G418. Cells are plated in 24-well plates coated with poly-L-lysine coverslips, at a density of 5×10⁵ cells/ml, for 24 hours prior to transfection in media lacking selection. Transfection conditions are optimized using LipofectAMINE 2000 (Invitrogen) at varying ratios of LipofectAMINE 2000 to oligonucleotide.

Cells are also treated with indicated oligonucleotides (see FIG. 18) until a 1 to 5 ratio is established. LF2000 is incubated with Opti-Mem I (serum-free medium) (Gibco BRL) for 5 minutes, the oligonucleotide is added, and incubation continued for 20 minutes at room temperature. The lipid/DNA mixture is applied to the cells at 37° C. for 12 hours, followed by fusion gene induction with 5 mM muristerone (Invitrogen Life technologies).

Protein aggregates are monitored for 72 hours post-transfection using a Zeiss inverted 100M Axioskop equipped with a Zeiss 510 LSM confocal microscope and a Coherent Krypton Argon laser and a Helium Neon laser. Samples are loaded into Lab-Tek II chambered coverglass system for improved imaging. The number of protein aggregates within at least five fields of view of the objective are counted, averaged, and standard deviation determined based on these numbers.

Results

Our strategy is based upon converting a single nucleotide in the polyglutamine repeat tract of the gene encoding the huntingtin protein. Oligonucleotides are designed to change one of the CAG repeats in exon 1 of the HD gene to CTG. Early attempts to use oligonucleotides consisting of the complementary sequence to the entire CAG repeat region failed to direct detectable single-base nucleotide alteration (data not shown). Thus, we amended the design so that the 5′ end of the oligonucleotide hybridized in the unique region of the first exon with only a part of the oligonucleotide being complementary to the CAG repeat region (see diagram, FIG. 19).

Lymphoblastoid cells containing 16 and 20 triplet repeat (CAG) alleles in the huntingtin (Htt) gene (as depicted in FIG. 19) are transfected with the oligonucleotide using the liposome DOTAP. The target is the second CAG repeat triplet, shown in bold in FIG. 19. Conversion of this nucleotide (A) to a T residue will create an RFLP that will enable cleavage by the enzyme PvuII.

To analyze for this event, the region surrounding and including the target base is amplified to generate a PCR product of 322 bases. The PCR fragment is ligated into a plasmid through the TA-cloning process (FIG. 20A) and propagated. The plasmid is isolated and then digested with PvuII; the restriction products are analyzed by gel electrophoresis.

The gels presented in FIGS. 20B and 20C are representative of the PCR products, first from genomic amplification (B) and then from the TA clones (C). As can be seen in FIG. 20B, a fragment of the predicted size is generated, and digestion of the TA cloned plasmid results in the appearance of cleaved products.

The frequency with which a new RFLP site is created, as evidenced by digestion with PvuII, is approximately 0.5%. This means that 1 out of 200 TA clones contains the converted/repaired sequence.

To confirm that the specific base is altered, a DNA sequence analysis is carried out. While the majority of clones contain the normal CAG repeat (FIG. 20D, upper panel), converted clones are found and exhibit the CTG codon in the second triplet position (FIG. 20D, lower panel).

The same experiment is repeated, substituting a 25-mer (HD3S/25) for the 52-mer (HD3S/52), and genomic DNA isolated from the transfected lymphoblastoid cells was amplified. The fragments are placed into plasmids by TA cloning and DNA sequence analyses carried out. As shown in FIG. 20D, clones containing a CTG codon at the targeted positions are obtained and, as in the case of the cells treated with the 52-mer, the frequency of these clones is approximately 0.5%.

Thus, within the context of this position and within exon I of Htt, the nucleotide exchange reaction appears to have a significant degree of specificity.

Nucleotide exchange can also be directed by double-stranded hairpin molecules known as chimeric RNA/DNA oligonucleotides (Figure FIG. 21A). These molecules contain complementary RNA and DNA residues folded into a double hairpin configuration and a single, unligated, phosphodiester bond to allow for topological intertwining upon hybridization at a designated target site. The mechanism by which chimeras direct a nucleotide exchange or “gene repair” event is likely to be similar to the pathway used by single-stranded DNA oligonucleotides.

Thus, the chimeric oligonucleotide is tested for nucleotide exchange activity, but using a different target site. In this case, the oligonucleotide is designed with the all-DNA strand being complementary to the long CAG repeat region. If successful, the nucleotide exchange reaction creates a stop codon, CAG to TAG. The mismatched base pair (see FIG. 21A), therefore, could occur at multiple sites within the target sequence. The strategy here is to expand the number of possible sites where nucleotide exchange could take place, thus targeting a larger region within the gene, rather than a specific unique sequence.

Lymphoblastoid cells are transfected with the chimeric RNA/DNA oligonucleotide using the lipofection reagent, LipofectAMINE. We find LipofectAMINE to be the most productive transfer vehicle for double-stranded DNA molecules. After six hours of incubation, the liposome solution is removed and the cells permitted to recover for 18 hours. The same procedure described above in this Example for DNA analyses from samples transfected with the single-stranded oligonucleotide is used to search for altered DNA sequences, except in this case an RFLP site would not be created.

Accordingly, we submit the genomic PCR fragments for DNA sequence analyses directly. As shown in FIG. 21B, the CAG repeat region is perfectly intact, except for a single position within the fourth CAG codon. At this unique site, a mixed peak comprised of several DNA residues is seen. Such a heterogeneous arrangement could indicate a population of genomic fragments differing in the nucleotide at that position.

The genomic PCR fragments are then cloned into separate plasmids and the region surrounding the site in question subjected to a second round of DNA sequence analyses. Two classes of DNA sequence are recovered. The first is identical to the target, unaltered sequence containing a perfect string of CAG codons. The second group, however, contains a T residue at the first position of the fourth codon, confirming the results of the genomic PCR heterogeneous population. Clones containing the TAG codon comprise approximately 1% of the total clones isolated and sequenced (data not shown), and no other CAG codon appears to be altered based on the sequence data from the genomic PCR or the isolated clone.

Taken together, these data suggest that targeted nucleotide exchange is possible within the CAG repeat of the Htt exon 1.

The results of nucleotide exchange in lymphoblastoid cells prompt us to carry out similar studies in a modified PC-12 cell line.

These cells have been stably transfected with an inducible truncated huntingtin-GFP fusion construct. The Htt fusion construct consists of the first 17 amino-terminal amino acids and 103 polyglutamine codons fused to eGFP at the carboxy-terminus of the encoded protein. The expression of the Htt fusion protein is under the control of a hormone-inducible promoter: addition of muristerone induces the transcription and the production of Htt containing a region of 103 polyglutamine residues. The presence of this protein leads to protein aggregation, which can be visualized by monitoring eGFP inside the cells.

The fusion gene in this cell line contains 103 polyglutamine (103Q) codons, repeated as groups of . . . CAACAGCAGCAACAGCAA . . . This sequence could confer enough unique sequence restriction to enable specific oligonucleotide recognition. Hence, a new oligonucleotide, HDA3S/53T, containing 53 nucleotide residues with three phosphorothioate linkages at each termini, is designed.

This PC-12 cell line has features that make it valuable in our study. Among them is the ability to monitor aggregate formation using eGFP expression as a marker/signal, since the fusion gene contains the first exon of Htt, 103Q repeats, and eGFP. As stated above, the expression of this gene is inducible, and thus we can test the effect of oligonucleotides on aggregate formation by transfecting the oligonucleotide prior to promoter activation.

The cells are maintained in low amounts of Zeocin and G418 and plated in 24-well plates coated with poly-L-lepine coverslips (E. Schweitzer, personal communication). HDA3S/53T is transfected using LF2000 and 24 hours later the cells are induced with muristerone. Protein aggregates appear after 24 hours and maximize in number after 48-72 hours. To examine the effect of HDA3S/53T on protein aggregation, the cells are viewed with a Zeiss inverted 100M Axioskop confocal microscope (510LSM) using a Coherent Krypton Argon and Helium Neon laser. For these studies, samples are loaded into the Lab-Tek II chambered coverglass system to improve image analyses. The number of protein aggregates is counted in seven independent, randomly-selected fields of view, and the results are presented in the panels displayed in FIG. 22.

Panels 22A and 22B represent the PC-12 cell lines 48 hours after exposure to muristerone. In each of these cases, the cells are treated with LF2000 to replicate the transfection conditions, but the liposomal carriers contained no oligonucleotides. Panel 22B provides a closer view of a control sample and helps illustrate the number of aggregates present inside the cells.

FIG. 22C illustrates the transfection efficiency of a Texas-red-labeled oligonucleotide. This oligonucleotide is introduced using LF2000 and is seen to co-localize with the fusion protein, in most cases blending with eGFP to produce a yellow color. These results indicate that the majority of PC-12 cells receive the oligonucleotide using these transfection conditions.

FIGS. 22D, 22E, and 22F represent fields of view of induced PC-12 cells that receive HDA3S/53T 24 hours prior to the addition of muristerone. In each case, the number of aggregates is diminished significantly and the green fluorescence is more equally distributed throughout the cell. Since the number of viable cells remains the same, transfection of oligonucleotides appears to have little negative impact on cell viability, but more of the cells appear to have a well-distributed green fluorescence. In our hands, these cells survive for approximately five days post induction; their loss is due in all likelihood to the continual accumulation of eGFP.

Using FIG. 22B as a standard, we estimate that approximately 60-70% of the untreated (with oligonucleotides) cells contain aggregates 48 hours after induction. This number can now be established as the standard and the number of aggregates in treated cells averaged from 4 fields of view (FIG. 18).

Since the reduction in aggregate/cell number is substantial with HDA3S/53T, we expand this assay to include cells treated with other nucleotides differing in length and/or chemical modification. The experimental protocol is the same as set forth above, and the number of cells containing aggregates are calculated in the same fashion.

As summarized in FIG. 18, reduction in aggregate number is obvious for cells treated by HDA3S/53T or HDA3S/53NT. HDA3S/53NT is the perfect complement of HDA3S/53T and has the sequence:

[SEQ ID NO:25]

5′ T*C*T*GTTGCTGCTGTTGTTGCTGTTGCAGCTGTTGGAAGGACTTG
AGGGAC*T*C*G 3′

(HDA3S/53NT)

The results indicate that strand of the Htt fusion gene targeted by the oligonucleotide does not in this case influence the degree of inhibition. A greater level of reduction is evident as the length of the oligonucleotide is shortened from 53 to 15 to 9, respectively. In addition, the results from the “Kan” series of non-specific oliognucleotides indicate that the drop in aggregate formation does not rely on the specific sequence of the Htt fusion gene being present in the oligonucleotide.

Molecules containing the full complement of phosphorothioate linkages are not effective in lowering aggregate number. To check for the value of such modifications contained in an oligonucleotide for the promotion of aggregate reduction, we utilize a base variation known as Locked Nucleic Acid (LNA). This modification involves the addition of a methylene bridge uniting the 2′ oxygen and the 4′ carbon. LNAs enable nuclease resistance while reducing the overall toxicity levels sometimes observed when chemically-modified, single-stranded DNA molecules are introduced into mammalian cells.

None of the oligonucleotides bearing 3, 4, or 25 LNA residues are found to reduce the number of cells containing aggregates. These data may indicate that the phosphorothioate linkage itself may be important in the inhibition process.

Subsequently, two oligonucleotides, Kan uR/25G and Kan uRD/25G, are tested, one of which is comprised entirely of 2′-O-methyl RNA residues, while the other contains three 2′-O-methyl RNA residues at each end.

A chimera consisting of a double-stranded paired RNA/DNA hybrid is ineffective in aggregate reduction.

Taken together, these data suggest that the most effective oligonucleotides for promoting the inhibition of aggregate formation or aggregate dispersal of single-stranded molecules with phosphorothioate linkages on each end ranging in length from 53 to 9 bases.

The data presented support the notion that specially-modified oligonucleotides can inhibit the formation of protein aggregates bearing Htt. But, the most significant challenge for a therapeutic molecule would be to disrupt protein aggregates already present in the cell.

In another series of experiments, we modify the protocol to test the influence of an oligonucleotide on pre-existing Htt aggregates. In this modified protocol, induction of polyglutamine (Q103) Htt expression precedes transfection of the oligonucleotide HDA3S/53T. Gene expression is induced 24 hours after seeding the cells and fluorescent protein aggregates are observed 24 hours later. A diverse population of aggregates were seen varying in size, shape, and number per cell.

Treatment with HDA3S/53T results in three major phenotypic responses within the cells.

Some cells containing aggregates appear unchanged and maintain the same appearance as controls. In other cells, the preformed aggregates are seen to diminish in size, fading into the surrounding cell matrix, detectable by the diffusion of the green fluorescence (FIG. 23A). And we also observe cells in which the absolute number of aggregates is reduced per cells (FIG. 23B); thus, a specific oligonucleotide can reduce the number of preformed aggregates in PC12 cells.

### EXAMPLE 8

### Administration of Single Stranded Oligonucleotides that are Specific or Non-Specific to the HD Gene to a Transgenic Animal Model System of HD Causes a Reduction of Huntingtin Protein Aggregates

An animal model system for Huntington's disease is obtained. See, e.g., Brouillet, Functional Neurology 15(4): 239-251 (2000), the disclosure of which is hereby incorporated by reference. See also Ona et al. Nature 399: 263-267 (1999), Bates et al. Hum Mol Genet. 6(10):1633-7 (1997) and Hansson et al. J. of Neurochemistry 78: 694-703, the disclosure of each of which is hereby incorporated by reference. See also Rubinsztein, D. C., Trends in Genetics, Vol. 18, No. 4, pp. 202-209 (a review on various animal and non-human models of HD), the disclosure of which is hereby incorporated by reference. For example, a transgenic mouse expressing human Huntingtin protein, a portion thereof, or fusion protein comprising human Huntingtin protein, or a portion thereof, with, for example, at least 36 CAG repeats (alternatively, any number of the CAG repeats may be CAA) in the CAG repeat segment of exon 1 encoding the poly Q tract. An example of such a transgenic mouse strain is the R6/2 line (Mangiarini et al. Cell 87: 493-506 (1996), the disclosure of which is hereby incorporated by reference). The R6/2 mice are transgenic Huntington's disease mice, which over-express exon one of the human HD gene (under the control of the endogenous promoter). The exon 1 of the R6/2 human HD gene has an expanded CAG/polyglutamine repeat lengths (150 CAG repeats on average). These mice develop a progressive, ultimately fatal neurological disease with many features of human Huntington's disease. Abnormal aggregates, constituted in part by the N-terminal part of Huntingtin (encoded by HD exon 1), are observed in R6/2 mice, both in the cytoplasms and nuclei of cells (Davies et al. Cell 90: 537-548 (1997)), the disclosure of which is hereby incorporated by reference). Preferably, the human Huntingtin protein in the transgenic animal has at least 55 CAG repeats and more preferably about 150 CAG repeats. These transgenic animals develop a Huntington's disease-like phenotype.

These transgenic mice are characterized by reduced weight gain and lifespan and motor impairment characterized by abnormal gait, resting tremor, hindlimb clasping and hyperactivity from 8 to 10 weeks after birth (for example the R6/2 strain; see Mangiarini et al. Cell 87: 493-506 (1996)). The phenotype worsens progressively toward hypokinesia. The brains of these transgenic mice also demonstrate neurochemical and histological abnormalities, such as changes in neurotransmitter receptors (glutamate, dopaminergic), decreased concentration of N-acetylaspartate (a marker of neuronal integrity) and reduced striatum and brain size. In addition, abnormal aggregates containing the transgenic part of or full-length human Huntingtin protein are present in the brain tissue of these animals. The R6/2 strain is an example of such a transgenic mouse strain. See Mangiarini et al. Cell 87: 493-506 (1996), Davies et al. Cell 90: 537-548 (1997), Brouillet, Functional Neurology 15(4): 239-251 (2000) and Cha et al. Proc. Natl. Acad. Sci. USA 95: 6480-6485 (1998).

To test the effect of the oligonucleotides described in the application in an animal model, different concentrations of HDA3T/53, or any other single stranded oligonucleotide or chimeric RNA/DNA oligonucleotide capable of causing an alteration in the HD gene (such as any of those described in this application, including in the Examples), or any oligonucleotide that can hybridize to the HD gene, or a single stranded oligonucleotide that is non-specific for HD (such as any of those described in Examples 2-3 and 6-8 or any of those single stranded oligonucleotide that is described in this application that is non-specific for the HD gene) are administered to the transgenic animal, for example by injecting pharmaceutical compositions comprising the oligonucleotides into the brain. The progression of the Huntington's disease-like symptoms, for example as described above for the mouse model, is then monitored to determine whether treatment with the oligonucleotides results in reduction or delay of symptoms. Alternatively, for example, disaggregation of the Huntingtin protein aggregates in these animals is monitored.

The animal is then sacrificed and brain slices are obtained. The brain slices are then analyzed for the presence of aggregates containing the transgenic human Huntingtin protein, a portion thereof, or fusion protein comprising human Huntingtin protein, or a portion thereof. This analysis includes, for example, staining the slices of brain tissue with anti-Huntingtin antibody and adding a secondary antibody conjugated with FITC which recognizes the anti-Huntingtin's antibody (for example, the anti-Huntingtin antibody is mouse anti-human antibody and the secondary antibody is specific for human antibody) and visualizing the protein aggregates by fluorescent microscopy. Alternatively, the anti-Huntingtin antibody can be directly conjugated with FITC. The levels of Huntingtin's protein aggregates are then visualized by fluorescent microscopy.

### EXAMPLE 9

### Determination of Possible Sequence Specificity of a Four Base Single Stranded Oligonucleotide in Causing Disaggregation of Huntingtin Aggregates

All 256 possible four base single stranded oligonucleotides are synthesized. These four base oligonucleotides may be modified by, for example, phosphorothioate linkage in one terminus or the other, or both, and/or one or more internal phosphorothioate linkages, or all phosphorothioate linkages. These 4 mers may be modified in any way as described in this application.

These 4 mer phosphorothioate oligonucleotides (which can be deoxyoligonucleotides or combinations of DNA with RNA, with LNA, or combinations of these) may be tested for their ability to cause disaggregation of huntingtin protein aggregates or treat Huntington's disease or symptoms in any in vitro or in vivo system, such as those described in Examples 1-7.

The results are evaluated to determine whether a 4 mer causes disaggregation of, or reduction in formation of, huntingtin protein aggregates or treat Huntington's disease or symptoms, and whether particular base sequences are better than others in causing disaggregation of, or reduction of the formation of, huntingtin protein aggregates or treating Huntington's disease or symptoms.

### EXAMPLE 10

### Administration of Single Stranded Oligonucleotides that are Specific or Non-Specific to the HD Gene to a Drosophila Model System of HD Causes a Reduction of Huntingtin Protein Aggregates

A Drosophila melanogaster model system for Huntington's disease is obtained. See, e.g., Steffan et al., Nature, 413: 739-743 (2001) and Marsh et al., Human Molecular Genetics 9: 13-25 (2000), the disclosure of each of which is hereby incorporated by reference. For example, a transgenic Drosophila expressing human Huntingtin protein, a portion thereof (such as exon 1), or fusion protein comprising human Huntingtin protein, or a portion thereof, with, for example, at least 36 CAG repeats (preferably 51 repeats or more) (alternatively, any number of the CAG repeats may be CAA) in the CAG repeat segment of exon 1 encoding the poly Q tract. These transgenic flies are engineered to express human Huntingtin protein, a portion thereof (such as exon 1), or fusion protein comprising human Huntingtin protein, or a portion thereof, in neurons.

To test the effect of the oligonucleotides described in the application in this Drosophila model, different concentrations of HDA3T/53, or any other single stranded oligonucleotide or chimeric RNA/DNA oligonucleotide capable of causing an alteration in the HD gene (such as any of those described in this application, including in the Examples), or any oligonucleotide that can hybridize to the HD gene, or a single stranded oligonucleotide that is non-specific for HD (such as any of those described in Examples 2-3 and 6-8 or any of those single stranded oligonucleotide that is described in this application that is non-specific for the HD gene) are administered to the transgenic Drosophila, for example, by injecting pharmaceutical compositions comprising the oligonucleotides into the brain, by orally administering the oligonucleotides, or by administering the oligonucleotides as part of food. Administration of the oligonucleotides can occur at various stages of the Drosophila life cycle. The progression of the Huntington's disease-like symptoms is then monitored to determine whether treatment with the oligonucleotides results in reduction or delay of symptoms. Alternatively, for example, disaggregation of the Huntingtin protein aggregates, or reduction in the formation of the Huntingtin protein aggregates in these flies is monitored. Alternatively, lethality and/or degeneration of photoreceptor neurons are monitored.

In fact, neurodegeneration due to expression of human Huntingtin protein, a portion thereof (such as exon 1), or fusion protein comprising human Huntingtin protein, or a portion thereof, is readily observed in the fly compound eye, which is composed of a regular trapezoidal arrangement of seven visible rhabdomeres (subcellular light-gathering structures) produced by the photoreceptor neurons of each Drosophila ommatidium. Expression of human Huntingtin protein, a portion thereof (such as exon 1), or fusion protein comprising human Huntingtin protein, or a portion thereof, leads to a progressive loss of rhabdomeres.

Results of administration of the oligonucleotides described in the application in this Drosophila model (such as different concentrations of HDA3T/53, or any other single stranded oligonucleotide or chimeric RNA/DNA oligonucleotide capable of causing an alteration in the HD gene (such as any of those described in this application, including in the Examples), or any oligonucleotide that can hybridize to the HD gene, or a single stranded oligonucleotide that is non-specific for HD (such as any of those described in Examples 2-3 and 6-8 or any of those single stranded oligonucleotide that is described in this application that is non-specific for the HD gene)) are evaluated to determine whether these oligonucleotides can, for example, retard or arrest neuronal degeneration.

### EXAMPLE 11

### Administration of Single Stranded Oligonucleotides that are Specific or Non-Specific to the HD Gene to an in vitro Model System of HD Causes a Reduction of Huntingtin Protein Aggregates

A microtiter plate assay for polyglutamine aggregate is obtained. See Berthelier et al., Analytical Biochemistry 295: 227-236 (2001), the disclosure of which is hereby incorporated by reference.

Following Berthelier et al., Analytical Biochemistry 295: 227-236 (2001), poly Q peptides of varying lengths are synthesized. Preferably, these peptides have pairs of Lys residues flanking the poly Q. The peptides can be biotinylated. The peptides can be about Q₂₈. An exemplary peptide is biotinylated K₂Q₃₀K₂. The peptides can be purified.

The peptides are solubilized and disaggregated by essentially the methods described in Berthelier et al., Analytical Biochemistry 295: 227-236 (2001). Poly Q aggregates are then formed from the solubilized peptides as described in Berthelier et al., Analytical Biochemistry 295: 227-236 (2001). The aggregates are collected by centrifugation, resuspended in a buffer (such as PBS, 0.01% Tween 20 and 0.05% NaN₃) and aliquoted into Eppendorf tubes. The tubes are snap-frozen in liquid nitrogen and stored at −80° C. Biotinylated peptides and aggregates of them are prepared essentially as described in Berthelier et al., Analytical Biochemistry 295: 227-236 (2001). 96-well microtiter plates with the aggregates in some or all the wells are prepared essentially as described in Berthelier et al., Analytical Biochemistry 295: 227-236 (2001). In some experiments, 20 ng per well of aggregates are used. Aggregate extension assays are done essentially as described in Berthelier et al., Analytical Biochemistry 295: 227-236 (2001).

The microtiter aggregate extension assay is used to test the ability of the oligonucleotides described in the application, including in the Examples (the oligonucleotides can be different concentrations of HDA3T/53, or any other single stranded oligonucleotide or chimeric RNA/DNA oligonucleotide capable of causing an alteration in the HD gene (such as any of those described in this application, including in the Examples), or any oligonucleotide that can hybridize to the HD gene, or a single stranded oligonucleotide that is non-specific for HD (such as any of those described in Examples 2-3 and 6-8 or any of those single stranded oligonucleotide that is described in this application that is non-specific for the HD gene)), to inhibit poly Q aggregate extension in this microtiter in vitro aggregate extension assay.

### EXAMPLE 12

### Use of a Yeast System to Determine HD Gene Alteration by Single Stranded Oligonucleotides

Specific gene conversion in yeast is analyzed. Two S. cerevisiae strains are provided: W303-1a (MAT a, Ade 2-1, trp 1-1, can 1-100, leu 2-3, 112 his 3-11, 15 ura 3-1) containing the first 170 codons of human HD with either 23 Q repeats (CAG (any of the CAG repeat may be CAA)) or 75 Q repeats, preferably constructed in such a way such that this portion of Huntingtin is expressed as a GFP fusion protein. Each of these strains bears the insert HD gene, preferably with an NLS (nuclear localization signal), under the control of an inducible promoter (Gal 1, 10) promoter or a constitutive (GPD-1) promoter. The portion of Huntingtin localizes to the nucleus and protein aggregates form in these cells. See Hughes et al., Proc. Natl. Acad. Sci. USA 98: 13201-13206 (2001), the disclosure of which is hereby incorporated by reference.

HD gene repair activity of any of the oligonucleotides described in the application, such as HDA3T/53, HDA3T/15 mer, HDA3T/9 mer, or any other single stranded oligonucleotide (such as any of those described in this application, including in the Examples, for example a 25 mer specific for repairing the CAG or CAA target site and containing one LNA on each end) is tested in this yeast system. Dosage levels and strandedness (strand bias for the template or non-template strand) of the oligonucleotides are tested. In some instances, the yeast cells are treated with hydroxyurea to reduce cell growth and extend the S phase of the cell cycle (higher efficiency targeting occur when the cells are in a prolonged S phase). In some instances, Trichostatin A (TSA) is added prior to the addition of the oligonucleotides. TSA and oligonucleotide together can have a synergistic effect on HD gene alteration.

Genetic conversion is carried out by dilution of the yeast in 96-well plates containing 10³ cells per well and conducting short DNA sequence analysis using an ABI SNAPSHOT automated sequencer. The capacity of this machine is 20-30 plates per week, and containing positive cells are expanded and are confirmed by subsequent direct DNA sequencing. The target site is within the HD gene CAG repeat; conversion of for example CAG to TAG is monitored. Huntingtin protein aggregate formation is also monitored (See Hughes et al., Proc. Natl. Acad. Sci. USA 98: 13201-13206 (2001)), using a Zeiss axiovert confocal microscope.

Equivalents

The invention may be embodied in other specific forms without departing from the spirit or essential characteristics thereof. The foregoing embodiments are therefore to be considered in all respects illustrative of, rather than limiting on, the invention disclosed herein.

All publications, patents and patent applications cited in this specification are herein incorporated by reference as if each individual publication, patent or patent application were specifically and individually indicated to be incorporated by reference.

We claim:

1. An oligonucleotide for targeted alteration of the genetic sequence of the Huntington's disease gene, comprising a single-stranded oligonucleotide having a DNA domain, said DNA domain having at least one mismatch with respect to the genetic sequence of the Huntington's disease gene to be altered, and further comprising chemical modifications of the oligonucleotide, said chemical modifications selected from the group consisting of an O-methyl modification, an LNA modification including LNA derivatives and analogs, two or more phosphorothioate linkages on one or more termini, and a combination of any two or more of these modifications.

2. The oligonucleotide according to claim 1, wherein said oligonucleotide comprises two or more phosphorothioate linkages on at least the 3′ terminus.

3. The oligonucleotide according to claim 1, wherein said oligonucleotide comprises one or more 2′-O-methyl analogs.

4. The oligonucleotide according to claim 1, wherein said oligonucleotide comprises an LNA nucleotide, including an LNA derivative or analog.

5. The oligonucleotide according to claim 1, wherein said oligonucleotide comprises a combination of at least two modifications selected from the group of a phosphorothioate linkage, a 2′-O-methyl analog, a locked nucleotide analog and a ribonucleotide.

6. The oligonucleotide according to claim 1, further comprising at least one unmodified ribonucleotide.

7. The oligonucleotide according to claim 2, wherein said oligonucleotide comprises two or more phosphorothioate linkages on both termini.

8. An oligonucleotide for targeted alteration of the genetic sequence of the Huntington's disease gene, comprising a chimeric RNA/DNA oligonucleotide, said oligonucleotide having at least one mismatch with respect to the genetic sequence of the Huntington's disease gene to be altered.

9. A method of targeted alteration of the genetic material of the Huntington's disease gene, comprising the step of combining the genetic material of the Huntington's disease gene with an oligonucleotide according to claim 1 or claim 8.

10. A method of targeted alteration of the genetic material of the Huntington's disease gene, comprising the step of administering to a cell extract an oligonucleotide of claim 1 or claim 8.

11. A method of targeted alteration of the genetic material of the Huntington's disease gene, comprising the step of administering to a cell an oligonucleotide of claim 1 or claim 8.

12. The method according to claim 11, wherein said genetic material of the Huntington's disease gene is a non-transcribed DNA strand of a duplex DNA.

13. An altered genetic material of the Huntington's disease gene obtained by the method of claim 10.

14. A cell comprising the altered genetic material of the Huntington's disease gene of claim 13.

15. A method of treating Huntington's disease, comprising the step of administering to a subject an effective amount of an oligonucleotide according to claim 1 or claim 8.

16. A method of prophylactically treating the severity of Huntington's disease, comprising the step of administering to a subject an effective amount of an oligonucleotide according to claim 1 or claim 8.

17. A method of inhibiting the formation of Huntingtin comprising protein aggregates in cells, said protein aggregates being a characteristic of Huntington's disease, comprising the step of administering to a subject an effective amount of an oligonucleotide according to claim 1 or claim 8.

18. A method of reducing Huntingtin comprising protein aggregates in cells, said protein aggregates being a characteristic of Huntington's disease, comprising the step of administering to a subject an effective amount of an oligonucleotide according to claim 1 or claim 8.

19. A method of treating Huntington's disease, comprising administering to a subject an effective amount of an oligonucleotide, wherein said oligonucleotide comprises a single-stranded oligonucleotide having a DNA domain, said DNA domain does or does not hybridize to the genetic sequence of the Huntington's disease gene, and further comprises chemical modifications of the oligonucleotide, said chemical modifications being selected from the group consisting of an o-methyl modification, an LNA modification including LNA derivatives and analogs, one or more phosphorothioate linkages on one or more termini, and a combination of any two or more of these modifications.

20. A method of preventing Huntington's disease, comprising the step of administering to a subject an effective amount of an oligonucleotide, wherein said oligonucleotide comprises a single-stranded oligonucleotide having a DNA domain, said DNA domain does or does not hybridize to the genetic sequence of the Huntington's disease gene, and further comprises chemical modifications of the oligonucleotide, said chemical modifications being selected from the group consisting of an o-methyl modification, an LNA modification including LNA derivatives and analogs, one or more phosphorothioate linkages on one or more termini, and a combination of any two or more of these modifications.

21. A method of reducing Huntingtin comprising protein aggregates in cells, said protein aggregates being a characteristic of Huntington's disease, comprising the step of administering to a subject an effective amount of an oligonucleotide, wherein said oligonucleotide comprises a single-stranded oligonucleotide having a DNA domain, said DNA domain does or does not hybridize to the genetic sequence of the Huntington's disease gene, and further comprises chemical modifications of the oligonucleotide, said chemical modifications being selected from the group consisting of an o-methyl modification, an LNA modification including LNA derivatives and analogs, one or more phosphorothioate linkages on one or more termini, and a combination of any two or more of these modifications.

22. A method of inhibiting the formation of Huntingtin comprising protein aggregates in cells, said protein aggregates being a characteristic of Huntington's disease, comprising the step of administering to a subject an effective amount of an oligonucleotide, wherein said oligonucleotide comprises a single-stranded oligonucleotide having a DNA domain, said DNA domain does or does not hybridize to the genetic sequence of the Huntington's disease gene, and further comprises chemical modifications of the oligonucleotide, said chemical modifications being selected from the group consisting of an o-methyl modification, an LNA modification including LNA derivatives and analogs, one or more phosphorothioate linkages on a terminus, and a combination of any two or more of these modifications.

23. The method according to any one of claims 19-22, wherein said oligonucleotide does not hybridize to the genetic sequence of the Huntington's disease gene.

24. The method according to any one of claims 19-22, wherein said oligonucleotide does hybridize to the genetic sequence of the Huntington's disease gene and wherein said DNA domain of said oligonucleotide has at least one mismatch with respect to the genetic sequence of the Huntington's disease gene to be altered.

25. The method according to any one of claims 19-22, wherein said oligonucleotide comprises one or more phosphorothioate linkages on at least the 3′ terminus.

26. The method according to claim 25, wherein said oligonucleotide comprises one or more phosphorothioate linkage on both termini.

27. The method according to claim 25, wherein said oligonucleotide comprises all phosphorothioate linkages.

28. The method according to any one of claims 19-22, wherein said oligonucleotide comprises a 2′-O-methyl analog.

29. The method according to any one of claims 19-22, wherein said oligonucleotide comprises a combination of at least two modifications selected from the group of a phosphorothioate linkage, a 2′-O-methyl analog, a locked nucleotide analog and a ribonucleotide.

30. The method according to any one of claims 19-22, wherein said oligonucleotide comprises at least one unmodified ribonucleotide.

31. The method according to claim 23, wherein said oligonucleotide comprises at least one unmodified ribonucleotide.

32. The method according to claim 24, wherein said oligonucleotide comprises at least one unmodified ribonucleotide.

33. The method according to any one of claims 19-22, wherein said oligonucleotide is about 4 nucleotides to about 25 nucleotides in length.

34. The method according to claim 33, wherein said oligonucleotide is about 4 nucleotides to about 15 nucleotides in length.

35. The method according to claim 33, wherein said oligonucleotide is about 4 nucleotides to about 9 nucleotides in length.
